(19) United States
(12) Patent Application Publication
(10) Pub. No.: US 2021/0247387 A1
(43) Pub. Date: Aug. 12, 2021

(54) IMPROVED PROTEOMIC MULTIPLEX ASSAYS

(71) Applicant: SOMALOGIC, INC., Boulder, CO (US)

(72) Inventors: Stephan KRAEMER, Boulder, CO (US); Evaldas KATILIUS, Superior, CO (US); Dominic ZICHI, Boulder, CO (US)

(73) Assignee: SOMALOGIC, INC., Boulder, CO (US)

(21) Appl. No.: 16/973,734

(22) PCT Filed: Jun. 19, 2019
(86) PCT No.: PCT/US2019/038022

§ 371 Date: Dec. 9, 2020

Related U.S. Application Data
(60) Provisional application No. 62/688,770, filed on Jun. 22, 2018.

(51) Int. Cl. G01N 33/53; C12Q 1/6848; G01N 33/68

(52) U.S. Cl. CPC G01N 33/5306; G01N 33/6803; C12Q 1/6848; G01N 33/5308

(57) ABSTRACT

Methods, devices, reagents and kits designed to improve the performance of proteomic based assays are provided. Such methods have a wide utility in proteomic applications for research and development, diagnostics and therapeutics by providing for a reduction or elimination of background signal and improved specificity for protein binding reagents in a multiplex assay formats.

### FIELD

The present disclosure relates generally to the field of proteomic assays, and methods, devices, reagents and kits designed to improve the performance of the assays. Such methods have a wide utility in proteomic applications for research and development, diagnostics and therapeutics. Specifically, materials and methods are provided for the reduction or elimination of background signal and improving the specificity of protein binding reagents in a multiplex assay format.

### BACKGROUND

Assays directed to the detection and quantification of physiologically significant molecules in biological samples and other sample types are important tools in scientific research and in the health care field. For example, multiplex array assays employ surface bound probes to detect target molecules in a sample. The surface-bound probes may be oligonucleotides, peptides, polypeptides, proteins, antibodies, affibodies, aptamers or other molecules (collectively biopolymers) capable of binding with target molecules from the sample. These binding interactions are the basis for many of the methods and devices used in a variety of different fields, e.g., genomics, transcriptomics and proteomics.

Assays provide solution-based target interaction and separation steps designed to remove specific components of an assay mixture. However, the sensitivity and specificity of many assay formats are limited by the ability of the detection method to resolve true signal from signal that arises due to nonspecific associations during the assay and result in a false detected signal. This is particularly true for multiplexed assays irrespective of the capture reagent used (e.g., antibody or aptamers). One of the key sources of non-specific binding is unanticipated non-specific capture reagent interactions with target molecules or non-specific binding interactions. This disclosure describes methods to eliminate or reduce the background signal observed in multiplexed based proteomic assay while maintaining target/capture reagent specific interactions.

### SUMMARY

In some embodiments, a method is disclosed which comprises a) contacting a first dilution sample with a first aptamer, wherein a first aptamer affinity complex is formed by the interaction of the first aptamer with its target molecule if the target molecule is present in the first dilution sample; b) contacting a second dilution sample with a second aptamer, wherein a second aptamer affinity complex is formed by the interaction of the second aptamer with its target molecule if the target molecule is present in the second dilution sample; c) incubating the first and second dilution samples separately to allow aptamer affinity complex formation; d) transferring the first dilution sample with the first aptamer affinity complex to a first mixture, wherein the first aptamer affinity complex is captured on a solid support in the first mixture; e) after step d), transferring the second dilution sample to the first mixture to form a second mixture, wherein the second aptamer affinity complex of the second dilution is captured on a solid support in the second mixture; f) detecting for the presence of or determining the level of the first aptamer and second aptamer of the first and second aptamer affinity complexes, or the presence or amount of one or more first and second aptamer affinity complexes; wherein, the first dilution and the second dilution are different dilutions of the same test sample.

In one aspect, the test sample is selected from plasma, serum, urine, whole blood, leukocytes, peripheral blood mononuclear cells, buffy coat, sputum, tears, mucus, nasal washes, nasal aspirate, semen, saliva, peritoneal washings, ascites, cystic fluid, meningeal fluid, amniotic fluid, glandular fluid, lymph fluid, nipple aspirate, bronchial aspirate, bronchial brushing, synovial fluid, joint aspirate, organ secretions, cells, a cellular extract, and cerebrospinal fluid.

In another aspect, the first and second aptamer-target molecule affinity complexes are non-covalent complexes.

In another aspect, the target molecule is selected from a protein, a peptide, a carbohydrate, a polysaccharide, a glycoprotein, a hormone, a receptor, an antigen, an antibody, a virus, a bacteria, a metabolite, a cofactor, an inhibitor, a drug, a dye, a nutrient, a growth factor, a cell and a tissue.

In another aspect, the first dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008% or is from 0.003% to 0.007% or is about 0.005%, and the second dilution is a dilution of the test sample of from 0.01% to 1% (or wherein is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8% or is from 0.2% to 0.75% or is about 0.5%.

In another aspect, the first dilution is a dilution of the test sample of from 0.001% to 0.009% (or 0.001%, 0.002%, 0.003%, 0.004%, 0.005%, 0.006%, 0.007%, 0.008% or 0.009%) or is from 0.002% to 0.008%, or is from 0.003% to 0.007% or is about 0.005%; and the second dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%.

In another aspect, the first dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%; and the second dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%.

In another aspect, the first dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%; and the second dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008%, or is from 0.003% to 0.007%, or is about 0.005%.

In another aspect, the first dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%, and the second dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%.

In another aspect, the first dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%, and the second dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008%, or is from 0.003% to 0.007%, or is about 0.005%.

In another aspect, the detecting for the presence or the determining of the level of the dissociated first and second capture reagents is performed by PCR, mass spectrometry, nucleic acid sequencing, next-generation sequencing (NGS) or hybridization.

In another aspect, the first aptamer and/or the second aptamer, independently, comprises at least one 5-position modified pyrimidine.

In another aspect, the at least one 5-position modified pyrimidine comprises a linker at the 5-position of the pyrimidine and a moiety attached to the linker.

In another aspect, the linker is selected from amide linker, a carbonyl linker, a propynyl linker, an alkyne linker, an ester linker, a urea linker, a carbamate linker, a guanidine linker, an amidine linker, a sulfoxide linker, and a sulfone linker.

In another aspect, wherein the moiety is a hydrophobic moiety.

In another aspect, the moiety is selected from the moieties of Groups I, II, III, IV, V, VII, VIII, IX, XI, XII, XIII, XV and XVI of FIG. 1.

In another aspect, the moiety is selected from a naphthyl moiety, a benzyl moiety, a fluorobenzyl moiety, a tyrosyl moiety, an indole moiety a morpholino moiety, an isobutyl moiety, a 3,4-methylenedioxy benzyl moiety, a benzothiophenyl moiety, and a benzofuranyl moiety.

In another aspect, the pyrimidine of the 5-position modified pyrimidine is a uridine, cytidine or thymidine.

In another aspect, the methods disclosed herein further comprise contacting a third dilution sample with a third aptamer, wherein a third aptamer affinity complex is formed by the interaction of the third aptamer with its target molecule if the target molecule is present in the third dilution sample;

In another aspect, the third dilution sample is incubated separately from the first and second dilution samples to allow aptamer affinity complex formation of the third aptamer with its target molecule.

In another aspect, the methods disclosed herein further comprise transferring the third dilution sample to the second mixture to form a third mixture, wherein the third aptamer affinity complex of the third dilution is captured on a solid support in the third mixture.

In another aspect, the methods disclosed herein further comprise detecting for the presence of or determining the level of the third aptamer of the third aptamer affinity complex, or the presence or amount of the third aptamer affinity complex;

In another aspect, the third dilution is a different dilution from the first dilution and the second dilution of the same test sample.

In another aspect, the third dilution is a dilution of the test sample selected from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), from 15% to 30%, from 15% to 25%, about 20%; from 0.01% to 1% (or 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%), from 0.1% to 0.8%, from 0.2% to 0.75%, about 0.5%; and from 0.001% to 0.009% (or 0.001%, 0.002%, 0.003%, 0.004%, 0.005%, 0.006%, 0.007%, 0.008% or 0.009%), or from 0.002% to 0.008%, from 0.003% to 0.007%, about 0.005%.

In another aspect, the third aptamer comprises at least one 5-position modified pyrimidine.

In another aspect, the at least one 5-positon modified pyrimidine comprises a linker at the 5-position of the pyrimidine and a moiety attached to the linker. In another aspect, the linker is selected from amide linker, a carbonyl linker, a propynyl linker, an alkyne linker, an ester linker, a urea linker, a carbamate linker, a guanidine linker, an amidine linker, a sulfoxide linker, and a sulfone linker.

In another aspect, the moiety is a hydrophobic moiety.

In another aspect, the moiety is selected from the moieties of Groups I, II, III, IV, V, VII, VIII, IX, XI, XII, XIII, XV and XVI of FIG. 1.

In another aspect, the moiety is selected from a naphthyl moiety, a benzyl moiety, a fluorobenzyl moiety, a tyrosyl moiety, an indole moiety a morpholino moiety, an isobutyl moiety, a 3,4-methylenedioxy benzyl moiety, a benzothiophenyl moiety, and a benzofuranyl moiety.

In another aspect, the pyrimidine of the 5-position modified pyrimidine is a uridine, cytidine or thymidine.

In some embodiments, a method is disclosed which comprises a) contacting a first capture reagent with a first dilution to form a first mixture and a second capture reagent with a second dilution to form a second mixture, wherein each of the first and second capture reagents are each immobilized on a solid support, and wherein each of the first and second capture reagents have affinity for a different target molecule; b) incubating the first mixture and the second mixture separately, wherein a first capture reagent-target molecule affinity complex is formed in the first mixture if the target molecule to which the first capture reagent has affinity for is present in the first mixture, wherein a second capture reagent-target molecule affinity complex is formed in the second mixture if the target molecule to which the second capture reagent has affinity for is present in the second mixture; c) sequentially releasing and combining the affinity complexes in a fourth mixture in an order selected from (i) the first capture reagent-target molecule affinity complex, followed by the second capture reagent-target molecule affinity complex and (ii) the second capture reagent-target molecule affinity complex, followed the first capture reagent-target molecule affinity complex; d) attaching a first tag to the target molecule of the first and second capture reagent-target molecule affinity complexes; e) contacting the tagged first and second capture reagent-target molecule affinity complexes to one or more solid supports such that the tag immobilizes the first and second capture reagent-target molecule affinity complexes to the one or more one solid supports; f) dissociating the capture reagents from the capture reagent-target molecule affinity complexes; g) detecting for the presence of or determining the level of the dissociated capture reagents; wherein, the first dilution and the second dilution are different dilutions of a test sample.

In one aspect, the test sample is selected from plasma, serum, urine, whole blood, leukocytes, peripheral blood mononuclear cells, buffy coat, sputum, tears, mucus, nasal washes, nasal aspirate, semen, saliva, peritoneal washings, ascites, cystic fluid, meningeal fluid, amniotic fluid, glandular fluid, lymph fluid, nipple aspirate, bronchial aspirate, bronchial brushing, synovial fluid, joint aspirate, organ secretions, cells, a cellular extract, and cerebrospinal fluid.

In one aspect, the first and second capture reagent-target protein affinity complexes are non-covalent complexes.

In one aspect, the first capture reagent and the second capture reagent are, independently, selected from an aptamer or an antibody.

In one aspect, the target molecule is selected from a protein, a peptide, a carbohydrate, a polysaccharide, a glycoprotein, a hormone, a receptor, an antigen, an antibody, a virus, a bacteria, a metabolite, a cofactor, an inhibitor, a drug, a dye, a nutrient, a growth factor, a cell and a tissue.

In one aspect, the first dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008% or is from 0.003% to 0.007% or is about 0.005%, and the second dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8% or is from 0.2% to 0.75% or is about 0.5%.

In one aspect, the first dilution is a dilution of the test sample of from 0.001% to 0.009% (or 0.001%, 0.002%, 0.003%, 0.004%, 0.005%, 0.006%, 0.007%, 0.008% or 0.009%) or is from 0.002% to 0.008%, or is from 0.003% to 0.007% or is about 0.005%; and the second dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%.

In one aspect, the first dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%; and the second dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%.

In one aspect, the first dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%; and the second dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008%, or is from 0.003% to 0.007%, or is about 0.005%.

In one aspect, the first dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%, and the second dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%.

In one aspect, the first dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%, and the second dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008%, or is from 0.003% to 0.007%, or is about 0.005%.

In one aspect, the detecting for the presence or the determining of the level of the dissociated first and second capture reagents is performed by PCR, mass spectrometry, nucleic acid sequencing, next-generation sequencing (NGS) or hybridization.

In another aspect, the methods disclosed herein further comprise contacting a third capture reagent with a third dilution to form a third mixture, wherein the third capture reagent is immobilized on a solid support, and wherein the third capture reagent has affinity for a different target molecule than the target molecules of the first and second capture reagents.

In another aspect, the methods disclosed herein further comprise incubating the third mixture separately from the first mixture and the second mixture, wherein a third capture reagent-target molecule affinity complex is formed in the third mixture if the target molecule to which the third capture reagent has affinity for is present in the third mixture.

In another aspect, the methods disclosed herein further comprise sequentially releasing and combining the third capture reagent-target molecule affinity with the first and second capture reagent-target molecule affinity complexes into the fourth mixture in an order selected from (i) the first capture reagent-target molecule affinity complex, followed by the second capture reagent-target molecule affinity complex, followed by the third capture reagent-target molecule affinity complex; (ii) the first capture reagent-target molecule affinity complex, followed by the third capture reagent-target molecule affinity complex, followed by the second capture reagent-target molecule affinity complex; (iii) the second capture reagent-target molecule affinity complex, followed by the third capture reagent-target molecule affinity complex, followed by the first capture reagent-target molecule affinity complex; (iv) the second capture reagent-target molecule affinity complex, followed by the first capture reagent-target molecule affinity complex, followed by the third capture reagent-target molecule affinity complex; (v) the third capture reagent-target molecule affinity complex, followed by the first capture reagent-target molecule affinity complex, followed by the second capture reagent-target molecule affinity complex; and (vi) the third capture reagent-target molecule affinity complex, followed by the second capture reagent-target molecule affinity complex, followed by the first capture reagent-target molecule affinity complex.

In one aspect, the third dilution is a different dilution from the first dilution and the second dilution of the same test sample.

In one aspect, the third dilution is a dilution of the test sample selected from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), from 15% to 30%, from 15% to 25%, about 20%; from 0.01% to 1% (or 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%), from 0.1% to 0.8%, from 0.2% to 0.75%, about 0.5%; and from 0.001% to 0.009% (or 0.001%, 0.002%, 0.003%, 0.004%, 0.005%, 0.006%, 0.007%, 0.008% or 0.009%), or from 0.002% to 0.008%, from 0.003% to 0.007%, about 0.005%.

In another aspect, the methods disclosed herein further comprise detecting for the presence of or determining the level of the third aptamer of the third aptamer affinity complex, or the presence or amount of the third aptamer affinity complex.

In one aspect, the aptamer comprises at least one 5-position modified pyrimidine.

In one aspect, the at least one 5-positon modified pyrimidine comprises a linker at the 5-position of the pyrimidine and a moiety attached to the linker.

In one aspect, the linker is selected from amide linker, a carbonyl linker, a propynyl linker, an alkyne linker, an ester linker, a urea linker, a carbamate linker, a guanidine linker, an amidine linker, a sulfoxide linker, and a sulfone linker.

In one aspect, the moiety is a hydrophobic moiety.

In one aspect, the moiety is selected from the moieties of Groups I, II, III, IV, V, VII, VIII, IX, XI, XII, XIII, XV and XVI of FIG. 1.

In one aspect, the moiety is selected from a naphthyl moiety, a benzyl moiety, a fluorobenzyl moiety, a tyrosyl moiety, an indole moiety a morpholino moiety, an isobutyl moiety, a 3,4-methylenedioxy benzyl moiety, a benzothiophenyl moiety, and a benzofuranyl moiety. In one aspect, the pyrimidine of the 5-position modified pyrimidine is a uridine, cytidine or thymidine.

In some embodiments, a method is disclosed which comprises a) contacting a first capture reagent with a first dilution to form a first mixture, a second capture reagent with a second dilution to form a second mixture, and a third capture reagent with a third dilution to form a third dilution mixture, wherein each of the first, second, and third capture reagents are each immobilized on a solid support, and wherein each of the first, second and third capture reagents have affinity for a different target molecule; b) incubating the first mixture, second mixture and third mixture separately, wherein a first capture reagent-target molecule affinity complex is formed in the first mixture if the target molecule to which the first capture reagent has affinity for is present in the first mixture, wherein a second capture reagent-target molecule affinity complex is formed in the second mixture if the target molecule to which the second capture reagent has affinity for is present in the second mixture, and wherein a third capture reagent-target molecule affinity complex is formed in the third mixture if the target molecule to which the third capture reagent has affinity for is present in the third mixture; c) sequentially releasing and combining the affinity complexes in a forth mixture in an order selected from (i) the first capture reagent-target molecule affinity complex, followed by the second capture reagent-target molecule affinity complex, followed by the third capture reagent-target molecule affinity complex; (ii) the first capture reagent-target molecule affinity complex, followed by the third capture reagent-target molecule affinity complex, followed by the second capture reagent-target molecule affinity complex; (iii) the second capture reagent-target molecule affinity complex, followed by the third capture reagent-target molecule affinity complex, followed by the first capture reagent-target molecule affinity complex; (iv) the second capture reagent-target molecule affinity complex, followed by the first capture reagent-target molecule affinity complex, followed by the third capture reagent-target molecule affinity complex; (v) the third capture reagent-target molecule affinity complex, followed by the first capture reagent-target molecule affinity complex, followed by the second capture reagent-target molecule affinity complex; and (vi) the third capture reagent-target molecule affinity complex, followed by the second capture reagent-target molecule affinity complex, followed by the first capture reagent-target molecule affinity complex; d) attaching a first tag to the target molecule of the first, second, and third capture reagent-target molecule affinity complexes; e) contacting the tagged first, second, and third capture reagent-target molecule affinity complexes to one or more solid supports such that the tag immobilizes the first, second and third capture reagent-target molecule affinity complexes to the one or more one solid supports; f) dissociating the capture reagents from the capture reagent-target molecule affinity complexes; g) detecting for the presence of or determining the level of the dissociated capture reagents; wherein, the first dilution, the second dilution, and third dilution are different dilutions of a test sample.

In one aspect, the test sample is selected from plasma, serum, urine, whole blood, leukocytes, peripheral blood mononuclear cells, buffy coat, sputum, tears, mucus, nasal washes, nasal aspirate, semen, saliva, peritoneal washings, ascites, cystic fluid, meningeal fluid, amniotic fluid, glandular fluid, lymph fluid, nipple aspirate, bronchial aspirate, bronchial brushing, synovial fluid, joint aspirate, organ secretions, cells, a cellular extract, and cerebrospinal fluid.

In one aspect, the first, second and third capture reagent-target protein affinity complexes are non-covalent complexes.

In one aspect, the first capture reagent, the second capture reagent and the third capture reagent are, independently, selected from an aptamer or an antibody.

In one aspect, the target molecule is selected from a protein, a peptide, a carbohydrate, a polysaccharide, a glycoprotein, a hormone, a receptor, an antigen, an antibody, a virus, a bacteria, a metabolite, a cofactor, an inhibitor, a drug, a dye, a nutrient, a growth factor, a cell and a tissue.

In one aspect, the detecting for the presence or the determining of the level of the dissociated first and second capture reagents is performed by PCR, mass spectrometry, nucleic acid sequencing, next-generation sequencing (NGS) or hybridization.

In one aspect, the aptamer comprises at least one 5-position modified pyrimidine.

In one aspect, the at least one 5-positon modified pyrimidine comprises a linker at the 5-position of the pyrimidine and a moiety attached to the linker.

In one aspect, the linker is selected from amide linker, a carbonyl linker, a propynyl linker, an alkyne linker, an ester linker, a urea linker, a carbamate linker, a guanidine linker, an amidine linker, a sulfoxide linker, and a sulfone linker.

In one aspect, the moiety is a hydrophobic moiety.

In one aspect, the moiety is selected from the moieties of Groups I, II, III, IV, V, VII, VIII, IX, XI, XII, XIII, XV and XVI of FIG. 1.

In one aspect, the moiety is selected from a naphthyl moiety, a benzyl moiety, a fluorobenzyl moiety, a tyrosyl moiety, an indole moiety a morpholino moiety, an isobutyl moiety, a 3,4-methylenedioxy benzyl moiety, a benzothiophenyl moiety, and a benzofuranyl moiety.

In one aspect, the pyrimidine of the 5-position modified pyrimidine is a uridine, cytidine or thymidine.

In one aspect, the first dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008% or is from 0.003% to 0.007% or is about 0.005%, the second dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8% or is from 0.2% to 0.75% or is about 0.5%; and the third dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%.

In one aspect, the first dilution is a dilution of the test sample of from 0.001% to 0.009% (or 0.001%, 0.002%, 0.003%, 0.004%, 0.005%, 0.006%, 0.007%, 0.008% or 0.009%) or is from 0.002% to 0.008%, or is from 0.003% to 0.007% or is about 0.005%; the second dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%; and the third dilution is a dilution of the test sample of from 0.01% to 1% (or wherein is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8% or is from 0.2% to 0.75% or is about 0.5%.

In one aspect, the first dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%; the second dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%; and the third dilution is a dilution of the test sample of from 0.001% to 0.009% (or 0.001%, 0.002%, 0.003%, 0.004%, 0.005%, 0.006%, 0.007%, 0.008% or 0.009%) or is from 0.002% to 0.008%, or is from 0.003% to 0.007% or is about 0.005%.

In one aspect, the first dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%; the second dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008%, or is from 0.003% to 0.007%, or is about 0.005%; and the third dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%.

In one aspect, the first dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%, the second dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%; and the third dilution is a dilution of the test sample of from 0.001% to 0.009% (or 0.001%, 0.002%, 0.003%, 0.004%, 0.005%, 0.006%, 0.007%, 0.008% or 0.009%) or is from 0.002% to 0.008%, or is from 0.003% to 0.007% or is about 0.005%.

In one aspect, the first dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%; the second dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008%, or is from 0.003% to 0.007%, or is about 0.005%; and the third dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8% or is from 0.2% to 0.75% or is about 0.5%.

The foregoing and other objects, features, and advantages of the invention will become more apparent from the following detailed description, which proceeds with reference to the accompanying figures.

### BRIEF DESCRIPTION OF THE DRAWINGS

FIG. 1. Certain exemplary 5-position modified uridines and cytidines that may be incorporated into aptamers.

FIG. 2. Certain exemplary modifications that may be present at the 5-position of uridine. The chemical structure of the C-5 modification includes the exemplary amide linkage that links the modification to the 5-position of the uridine. The 5-position moieties shown include a benzyl moiety (e.g., Bn, PE and a PP), a naphthyl moiety (e.g., Nap, 2Nap, NE), a butyl moiety (e.g, iBu), a fluorobenzyl moiety (e.g., FBn), a tyrosyl moiety (e.g., a Tyr), a 3,4-methylenedioxy benzyl (e.g., MBn), a morpholino moiety (e.g., MOE), a benzofuranyl moiety (e.g., BF), an indole moiety (e.g, Trp) and a hydroxypropyl moiety (e.g., Thr).

FIG. 3. Certain exemplary modifications that may be present at the 5-position of cytidine. The chemical structure of the C-5 modification includes the exemplary amide linkage that links the modification to the 5-position of the cytidine. The 5-position moieties shown include a benzyl moiety (e.g., Bn, PE and a PP), a naphthyl moiety (e.g., Nap, 2Nap, NE, and 2NE) and a tyrosyl moiety (e.g., a Tyr).

FIG. 4. Provides an example overview of the dilution sets for a biological sample, the corresponding capture reagent sets for their respective dilutions, and the general overview of the two-catch system (catch-1 and catch-2). Two different dilution groups may be created from a biological sample that includes a Z% dilution of the biological sample or DIL4 and an X% dilution of the biological sample or DIL1, where Z is greater than X (or Z is a greater dilution than the X dilution). Each dilution has its own set of corresponding capture reagents (A3 for DIL1 and A1 for DIL4) that bind to a specific set of proteins. The two different dilution sets were transferred together from the catch-1 step of the assay to the catch-2 step of the assay. FIG. 5. Provides an example overview of the dilution sets for a biological sample, the corresponding capture reagent sets for their respective dilutions, and the general overview of the two-catch system (catch-1 and catch-2). Three different dilution groups may be created from a biological sample that includes a Z% dilution of the biological sample or DIL3, a Y% dilution of the biological sample or DIL2 and a X% dilution of the biological sample or DIL1, where Z is greater than Y, and Y is greater than X (or Z is a greater dilution than the Y dilution, and the Y dilution is a greater dilution than the X dilution). Each dilution has its own set of corresponding capture reagents (A3 for DIL1 A2 for DIL2 and A1 for DIL3) that bind to a specific set of proteins.

FIG. 6. Provides an overview of the three different dilution groups of plasma that were made: a 0.005% dilution (DIL1), a 0.5% dilution (DIL2) and a 20% dilution (DIL3), where the relative high, medium and low abundance proteins were measured, respectively. Further, the aptamer sets for each of DIL1, DIL2 and DIL3 were A1, A2 and A3, respectively. The A3 group of aptamers had 4,271 different aptamers (or ˜81% of the total number of aptamers), the A2 group had 828 different aptamers (or ˜16% of the total number of aptamers) and the A1 group has 173 different aptamers (˜3% of the total number of aptamers) for a total of 5,272 different aptamers. The three different dilution sets were transferred together from the catch-1 step of the assay to the catch-2 step of the assay.

FIG. 7. Provides an example overview of the dilution sets for a biological sample, the corresponding capture reagent sets for their respective dilutions, and the general overview of the sequential two-catch system (catch-1 and catch-2). Three different dilution groups may be created from a biological sample that includes a Z% dilution of the biological sample or DIL3, a Y% dilution of the biological sample or DIL2 and a X% dilution of the biological sample or DIL1, where Z is greater than Y, and Y is greater than X (or Z is a greater dilution than the Y dilution, and the Y dilution is a greater dilution than the X dilution). Each dilution has its own set of corresponding capture reagents (A3 for DIL1 A2 for DIL2 and A1 for DIL3) that bind to a specific set of proteins.

FIG. 8. Provides an overview of the three different dilution groups of plasma that were made: a 0.005% dilution (DIL1), a 0.5% dilution (DIL2) and a 20% dilution (DIL3), where the relative high, medium and low abundance proteins were measured, respectively. Further, the aptamer sets for each of DIL1 DIL2 and DIL3 were A1, A2 and A3, respectively. The A3 group of aptamers had 4,271 different aptamers (or ˜81% of the total number of aptamers), the A2 group had 828 different aptamers (or ˜16% of the total number of aptamers) and the A1 group has 173 different aptamers (-3% of the total number of aptamers) for a total of 5,272 different aptamers. The three different dilution sets were transferred sequentially from the catch-1 step of the assay to the catch-2 step of the assay.

FIG. 9. Provides an example overview of the dilution sets for a biological sample, the corresponding capture reagent sets for their respective dilutions, and the general overview of the two-catch system (catch-1 and catch-2). Two different dilution groups may be created from a biological sample that includes a Z% dilution of the biological sample or DIL4 and an X% dilution of the biological sample or DIL1, where Z is greater than X (or Z is a greater dilution than the X dilution). Each dilution has its own set of corresponding capture reagents (A3 for DIL1 and A1 for DIL4) that bind to a specific set of proteins. The two different dilution sets were transferred sequentially from the catch-1 step of the assay to the catch-2 step of the assay. FIG. 10. The cumulative distribution function (CDF) of the ratio of the aptamer signal for Condition 1 (i.e., all three dilution groups DIL1, DIL2 and DIL3) to the aptamer signal for each of Conditions 2, 3 and 4 (Table 2; where only one of the dilution groups was present along with blanks) was plotted for the assay as performed where all three dilution sets were transferred together from the catch-1 part of the assay to the catch-2 part of the assay. The ratio of aptamer signals are represented by relative fluorescent units (RFU's) derived from a hybridization array. FIG. 11. The cumulative distribution function (CDF) of the ratio of the aptamer signal for Condition 1 (i.e., all three dilution groups DIL1 DIL2 and DIL3) to the aptamer signal for each of Conditions 2, 3 and 4 (where only one of the dilution groups was present along with blanks) was plotted for the assay as performed where the three dilution sets were transferred sequentially from the catch-1 part of the assay to the catch-2 part of the assay. The ratio of aptamer signals are represented by relative fluorescent units (RFU's) derived from a hybridization array FIG. 12. A graphical representation of the number of analytes in the linear range (Y-axis; right side) along with the Median S/B (Y-axis; left side) for each of the dilutions of 40%, 20%, 10% and 5% (X-axis). At the 20% dilution of the biological sample, the maximum number of analytes in the linear range having the greatest Median S/B is observed (where the two lines intersect).

### DETAILED DESCRIPTION

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Further, ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of” mean ±20% of the indicated range, value, or structure, unless otherwise indicated. As used herein, the terms “include” and “comprise” are open ended and are used synonymously.

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

As used herein, the term “nucleotide” refers to a ribonucleotide or a deoxyribonucleotide, or a modified form thereof, as well as an analog thereof. Nucleotides include species that include purines (e.g., adenine, hypoxanthine, guanine, and their derivatives and analogs) as well as pyrimidines (e.g., cytosine, uracil, thymine, and their derivatives and analogs). As used herein, the term “cytidine” is used generically to refer to a ribonucleotide, deoxyribonucleotide, or modified ribonucleotide comprising a cytosine base, unless specifically indicated otherwise. The term “cytidine” includes 2′-modified cytidines, such as 2′-fluoro, 2′-methoxy, etc. Similarly, the term “modified cytidine” or a specific modified cytidine also refers to a ribonucleotide, deoxyribonucleotide, or modified ribonucleotide (such as 2′-fluoro, 2′-methoxy, etc.) comprising the modified cytosine base, unless specifically indicated otherwise. The term “uridine” is used generically to refer to a ribonucleotide, deoxyribonucleotide, or modified ribonucleotide comprising a uracil base, unless specifically indicated otherwise. The term “uridine” includes 2′-modified uridines, such as 2′-fluoro, 2′-methoxy, etc. Similarly, the term “modified uridine” or a specific modified uridine also refers to a ribonucleotide, deoxyribonucleotide, or modified ribonucleotide (such as 2′-fluoro, 2′-methoxy, etc.) comprising the modified uracil base, unless specifically indicated otherwise.

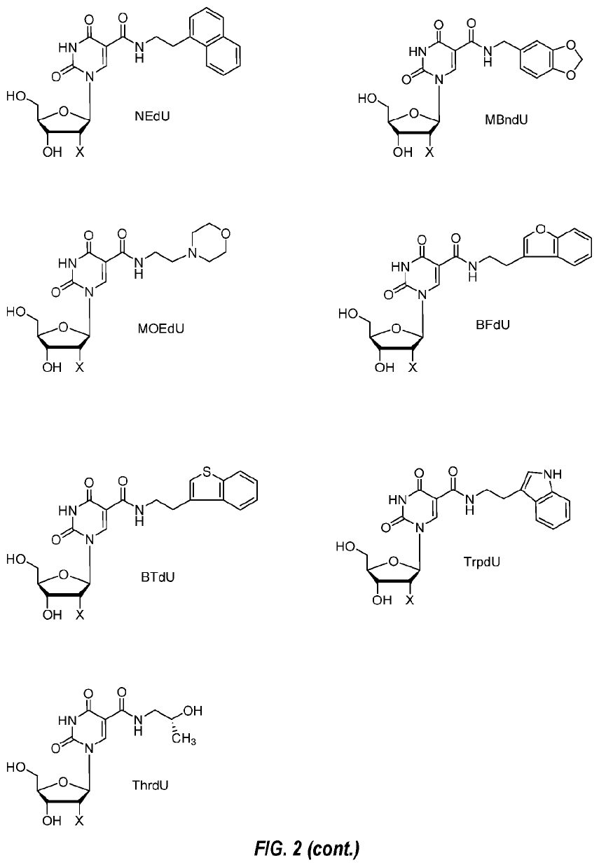

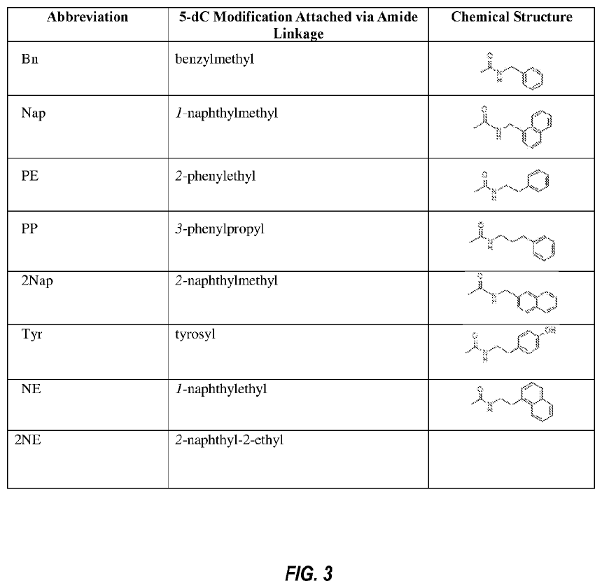

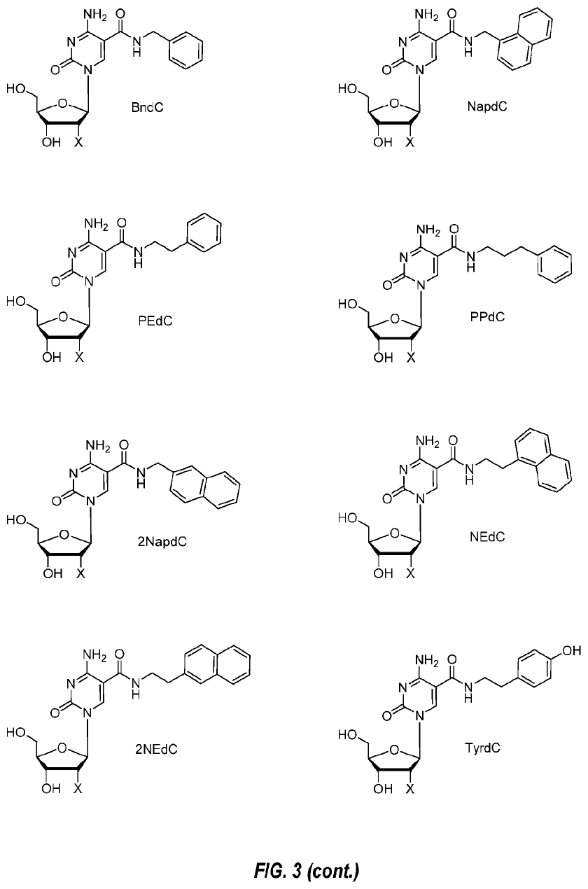

As used herein, the term “C-5 modified carboxamidecytidine” or “cytidine-5-carboxamide” or “5-position modified cytidine” or “C-5 modified cytidine” refers to a cytidine with a carboxyamide (—C(O)NH—) modification at the C-5 position of the cytidine including, but not limited to, those moieties (R^{X1}) illustrated herein. Exemplary C-5 modified carboxamidecytidines include, but are not limited to, 5-(N-benzylcarboxamide)-2′-deoxycytidine (referred to as “BndC” and shown in FIG. 3); 5-(N-2-phenylethylcarboxamide)-2′-deoxycytidine (referred to as “PEdC” and shown in FIG. 3); 5-(N-3-phenylpropylcarboxamide)-2′-deoxycytidine (referred to as “PPdC” and shown in FIG. 3); 5-(N-1-naphthylmethylcarboxamide)-2′-deoxycytidine (referred to as “NapdC” and shown in FIG. 3); 5-(N-2-naphthylmethylcarboxamide)-2′-deoxycytidine (referred to as “2NapdC” and shown in FIG. 3); 5-(N-1-naphthyl-2-ethylcarboxamide)-2′-deoxycytidine (referred to as “NEdC” and shown in FIG. 3); 5-(N-2-naphthyl-2-ethylcarboxamide)-2′-deoxycytidine (referred to as “2NEdC” and shown in FIG. 3); and 5-(N-tyrosylcarboxyamide)-2′-deoxycytidine (referred to as TyrdC and shown in FIG. 3). In some embodiments, the C5-modified cytidines, e.g., in their triphosphate form, are capable of being incorporated into an oligonucleotide by a polymerase (e.g., KOD DNA polymerase).

Chemical modifications of the C-5 modified cytidines described herein can also be combined with, singly or in any combination, 2′-position sugar modifications, modifications at exocyclic amines, and substitution of 4-thiocytidine and the like. As used herein, the term “C-5 modified carboxamidecytosine” or “cytosine-5-carboxamide” or “5-position modified cytosine” or “C-5 modified cytosine” refers to a cytosine base with a carboxyamide (—C(O)NH—) modification at the C-5 position of the cytosine including, but not limited to, those moieties (R^{X1}) illustrated herein. Exemplary C-5 modified carboxamidecytosines include, but are not limited to, the modified cytidines shown in FIG. 3.

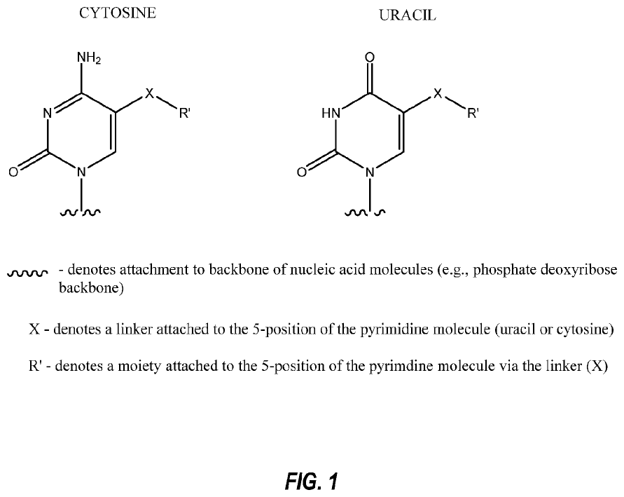

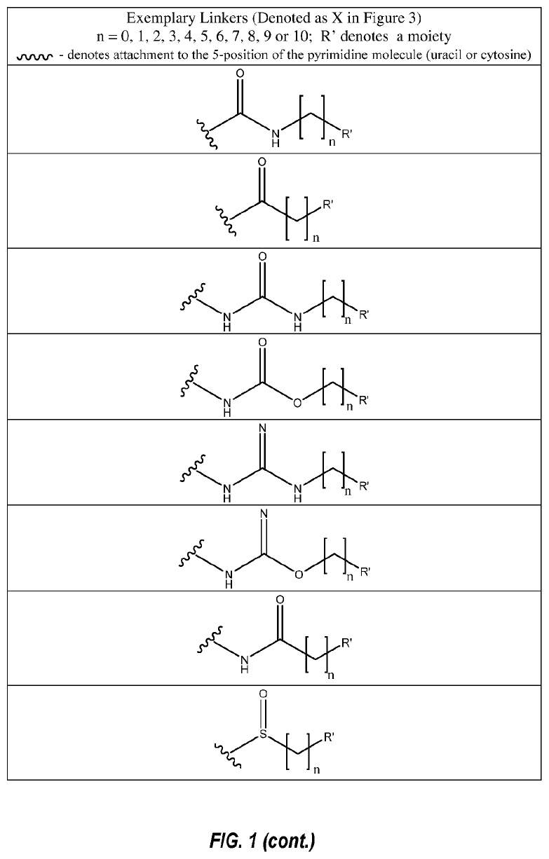

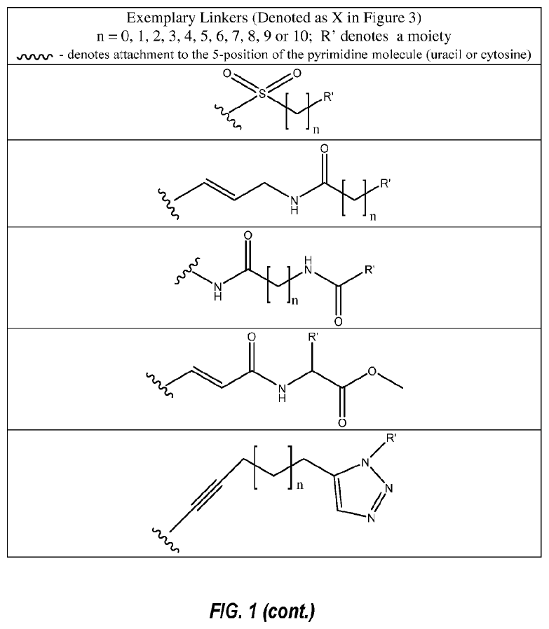

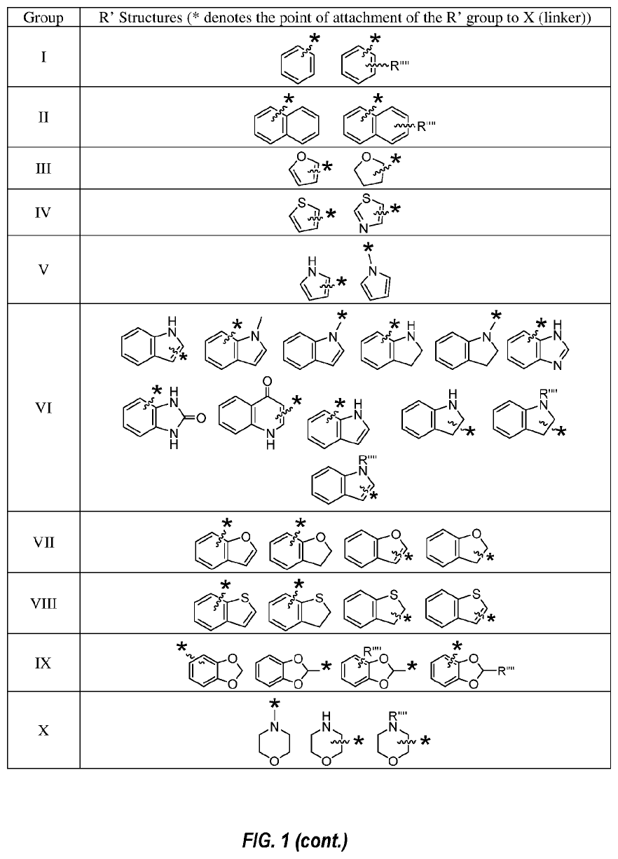

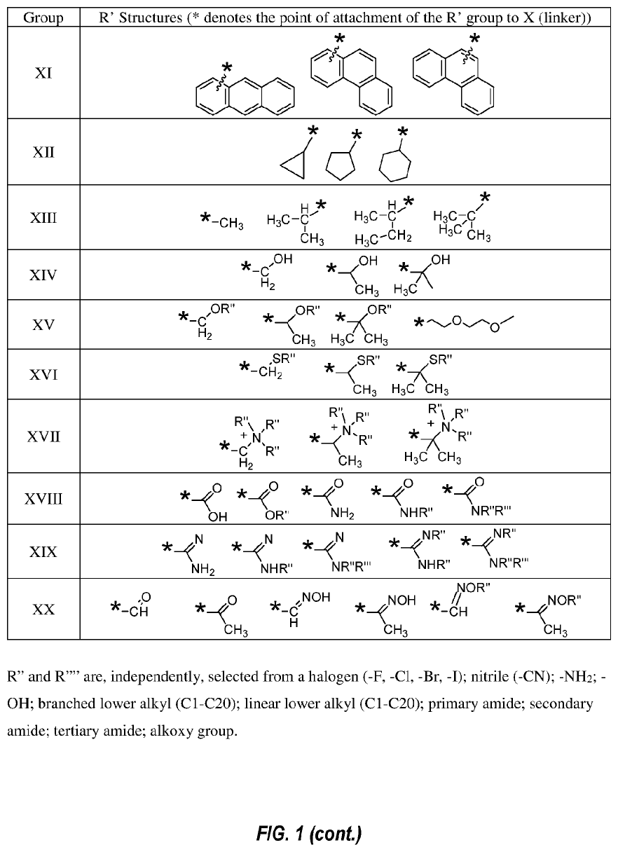

As used herein, the term “C-5 modified uridine” or “5-position modified uridine” refers to a uridine (typically a deoxyuridine) with a carboxyamide (—C(O)NH—) modification at the C-5 position of the uridine, e.g., as shown in FIG. 1. In some embodiments, the CS-modified uridines, e.g., in their triphosphate form, are capable of being incorporated into an oligonucleotide by a polymerase (e.g., KOD DNA polymerase). Nonlimiting exemplary 5-position modified uridines include:

5-(N-benzylcarboxyamide)-2′-deoxyuridine (BndU),

5-(N-benzylcarboxyamide)-2′-O-methyluridine,

5-(N-benzylcarboxyamide)-2′-fluorouridine,

5-(N-phenethylcarboxyamide)-2′-deoxyuridine (PEdU),

5-(N-thiophenylmethylcarboxyamide)-2′-deoxyuridine (ThdU),

5-(N-isobutylcarboxyamide)-2′-deoxyuridine (iBudU),

5-(N-tyrosylcarboxyamide)-2′-deoxyuridine (TyrdU),

5-(N-3,4-methylenedioxybenzylcarboxyamide)-2′-deoxyuridine (MBndU),

5-(N-4-fluorobenzylcarboxyamide)-2′-deoxyuridine (FBndU),

5-(N-3-phenylpropylcarboxyamide)-2′-deoxyuridine (PPdU),

5-(N-imidizolylethylcarboxyamide)-2′-deoxyuridine (ImdU),

5-(N-isobutylcarboxyamide)-2′-O-methyluridine,

5-(N-isobutylcarboxyamide)-2′-fluorouridine,

5-(N-tryptaminocarboxyamide)-2′-deoxyuridine (TrpdU),

5-(N-R-threoninylcarboxyamide)-2′-deoxyuridine (ThrdU),

5-(N-tryptaminocarboxyamide)-2′-O-methyluridine,

5-(N-tryptaminocarboxyamide)-2′-fluorouridine,

5-(N-[1-(3-trimethylamonium) propyl] carboxyamide)-2′-deoxyuridine chloride,

5-(N-naphthylmethylcarboxyamide)-2′-deoxyuridine (NapdU),

5-(N-naphthylmethylcarboxyamide)-2′-O-methyluridine,

5-(N-naphthylmethylcarboxyamide)-2′-fluorouridine,

5-(N-[1-(2,3-dihydroxypropyl)]carboxyamide)-2′-deoxyuridine),

5-(N-2-naphthylmethylcarboxyamide)-2′-deoxyuridine (2NapdU),

5-(N-2-naphthylmethylcarboxyamide)-2′-O-methyluridine,

5-(N-2-naphthylmethylcarboxyamide)-2′-fluorouridine,

5-(N-1-naphthylethylcarboxyamide)-2′-deoxyuridine (NEdU),

5-(N-1-naphthylethylcarboxyamide)-2′-O-methyluridine,

5-(N-1-naphthylethylcarboxyamide)-2′-fluorouridine,

5-(N-2-naphthylethylcarboxyamide)-2′-deoxyuridine (2NEdU),

5-(N-2-naphthylethylcarboxyamide)-2′-O-methyluridine,

5-(N-2-naphthylethylcarboxyamide)-2′-fluorouridine,

5-(N-3-benzofuranylethylcarboxyamide)-2′-deoxyuridine (BFdU),

5-(N-3-benzofuranylethylcarboxyamide)-2′-O-methyluridine,

5-(N-3-benzofuranylethylcarboxyamide)-2′-fluorouridine,

5-(N-3-benzothiophenylethylcarboxyamide)-2′-deoxyuridine (BTdU),

5-(N-3-benzothiophenylethylcarboxyamide)-2′-O-methyluridine, and

5-(N-3-benzothiophenylethylcarboxyamide)-2′-fluorouridine.

As used herein, the terms “modify,” “modified,” “modification,” and any variations thereof, when used in reference to an oligonucleotide, means that at least one of the four constituent nucleotide bases (i.e., A, G, T/U, and C) of the oligonucleotide is an analog or ester of a naturally occurring nucleotide. In some embodiments, the modified nucleotide confers nuclease resistance to the oligonucleotide. Additional modifications can include backbone modifications, methylations, unusual base-pairing combinations such as the isobases isocytidine and isoguanidine, and the like. Modifications can also include 3′ and 5′ modifications, such as capping. Other modifications can include substitution of one or more of the naturally occurring nucleotides with an analog, internucleotide modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present on the sugar of a nucleotide may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, polyethylene glycol (PEG) polymers in one embodiment ranging from about 10 to about 80 kDa, PEG polymers in another embodiment ranging from about 20 to about 60 kDa, or other hydrophilic or hydrophobic biological or synthetic polymers.

As used herein, “nucleic acid,” “oligonucleotide,” and “polynucleotide” are used interchangeably to refer to a polymer of nucleotides and include DNA, RNA, DNA/RNA hybrids and modifications of these kinds of nucleic acids, oligonucleotides and polynucleotides, wherein the attachment of various entities or moieties to the nucleotide units at any position are included. The terms “polynucleotide,” “oligonucleotide,” and “nucleic acid” include double- or single-stranded molecules as well as triple-helical molecules. Nucleic acid, oligonucleotide, and polynucleotide are broader terms than the term aptamer and, thus, the terms nucleic acid, oligonucleotide, and polynucleotide include polymers of nucleotides that are aptamers but the terms nucleic acid, oligonucleotide, and polynucleotide are not limited to aptamers.

Polynucleotides can also contain analogous forms of ribose or deoxyribose sugars that are generally known in the art, including 2′-O-methyl, 2′-O-allyl, 2′-O-ethyl, 2′-O-propyl, 2′-O-CH₂CH₂OCH₃, 2′-fluoro, 2′-NH₂ or 2′-azido, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. As noted herein, one or more phosphodiester linkages may be replaced by alternative linking groups. These alternative linking groups include embodiments wherein phosphate is replaced by P(O)S (“thioate”), P(S)S (“dithioate”), (O)NR^{X} 2 (“amidate”), P(O) R^{X}, P(O)OR ^{X}′, CO or CH₂ (“formacetal”), in which each R^{x} or R^{X}' are independently H or substituted or unsubstituted alkyl (C1-C20) optionally containing an ether (—O—) linkage, aryl, alkenyl, cycloalky, cycloalkenyl or araldyl. Not all linkages in a polynucleotide need be identical. Substitution of analogous forms of sugars, purines, and pyrimidines can be advantageous in designing a final product, as can alternative backbone structures like a polyamide backbone, for example. Polynucleotides can also contain analogous forms of carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside.

If present, a modification to the nucleotide structure can be imparted before or after assembly of a polymer. A sequence of nucleotides can be interrupted by non-nucleotide components. A polynucleotide can be further modified after polymerization, such as by conjugation with a labeling component.

As used herein, the term “at least one nucleotide” when referring to modifications of a nucleic acid, refers to one, several, or all nucleotides in the nucleic acid, indicating that any or all occurrences of any or all of A, C, T, G or U in a nucleic acid may be modified or not.

As used herein, “nucleic acid ligand,” “aptamer,” “SOMAmer,” “modified aptamer,” and “clone” are used interchangeably to refer to a non-naturally occurring nucleic acid that has a desirable action on a target molecule. A desirable action includes, but is not limited to, binding of the target, catalytically changing the target, reacting with the target in a way that modifies or alters the target or the functional activity of the target, covalently attaching to the target (as in a suicide inhibitor), and facilitating the reaction between the target and another molecule. In one embodiment, the action is specific binding affinity for a target molecule, such target molecule being a three dimensional chemical structure other than a polynucleotide that binds to the aptamer through a mechanism which is independent of Watson/Crick base pairing or triple helix formation, wherein the aptamer is not a nucleic acid having the known physiological function of being bound by the target molecule. Aptamers to a given target include nucleic acids that are identified from a candidate mixture of nucleic acids, where the aptamer is a ligand of the target, by a method comprising: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers of the target molecule are identified. It is recognized that affinity interactions are a matter of degree; however, in this context, the “specific binding affinity” of an aptamer for its target means that the aptamer binds to its target generally with a much higher degree of affinity than it binds to other, non-target, components in a mixture or sample. An “aptamer,” “SOMAmer,” or “nucleic acid ligand” is a set of copies of one type or species of nucleic acid molecule that has a particular nucleotide sequence. An aptamer can include any suitable number of nucleotides. “Aptamers” refer to more than one such set of molecules. Different aptamers can have either the same or different numbers of nucleotides. Aptamers may be DNA or RNA and may be single stranded, double stranded, or contain double stranded or triple stranded regions. In some embodiments, the aptamers are prepared using a SELEX process as described herein, or known in the art.

As used herein, a “SOMAmer” or Slow Off-Rate Modified Aptamer refers to an aptamer having improved off-rate characteristics. SOMAmers can be generated using the improved SELEX methods described in U.S. Pat. No. 7,947,447, entitled “Method for Generating Aptamers with Improved Off-Rates.”

As used herein, an aptamer comprising two different types of 5-position modified pyrimidines or C-5 modified pyrimidines may be referred to as “dual modified aptamers”, aptamers having “two modified bases”, aptamers having “two base modifications” or “two bases modified”, aptamer having “double modified bases”, all of which may be used interchangeably. A library of aptamers or aptamer library may also use the same terminology. Thus, in some embodiments, an aptamer comprises two different 5-position modified pyrimidines wherein the two different 5-position modified pyrimidines are selected from a NapdC and a NapdU, a NapdC and a PPdU, a NapdC and a MOEdU, a NapdC and a TyrdU, a NapdC and a ThrdU, a PPdC and a PPdU, a PPdC and a NapdU, a PPdC and a MOEdU, a PPdC and a TyrdU, a PPdC and a ThrdU, a NapdC and a 2NapdU, a NapdC and a TrpdU, a 2NapdC and a NapdU, and 2NapdC and a 2NapdU, a 2NapdC and a PPdU, a 2NapdC and a TrpdU, a 2NapdC and a TyrdU, a PPdC and a 2NapdU, a PPdC and a TrpdU, a PPdC and a TyrdU, a TyrdC and a TyrdU, a TrydC and a 2NapdU, a TyrdC and a PPdU, a TyrdC and a TrpdU, a TyrdC and a TyrdU, and a TyrdC and a TyrdU. In some embodiments, an aptamer comprises at least one modified uridine and/or thymidine and at least one modified cytidine, wherein the at least one modified uridine and/or thymidine is modified at the 5-position with a moiety selected from a naphthyl moiety, a benzyl moiety, a fluorobenzyl moiety, a tyrosyl moiety, an indole moiety a morpholino moiety, an isobutyl moiety, a 3,4-methylenedioxy benzyl moiety, a benzothiophenyl moiety, and a benzofuranyl moiety, and wherein the at least one modified cytidine is modified at the 5-position with a moiety selected from a naphthyl moiety, a tyrosyl moiety, and a benzyl moiety. In certain embodiments, the moiety is covalently linked to the 5-position of the base via a linker comprising a group selected from an amide linker, a carbonyl linker, a propynyl linker, an alkyne linker, an ester linker, a urea linker, a carbamate linker, a guanidine linker, an amidine linker, a sulfoxide linker, and a sulfone linker. See FIG. 1 for further examples of exemplary linkers that may be used to covalently link a moiety to the 5-position of a pyrimidine.

As used herein, a “hydrophobic group” and “hydrophobic moiety” are used interchangeably herein and refer to any group or moiety that is uncharged, a majority of the atoms of the group or moiety are hydrogen and carbon, the group or moiety has a small dipole and/or the group or moiety tends to repel from water. These groups or moeities may comprise an aromatic hydrocarbon or a planar aromatic hydrocarbon. Methods for determining the hydrophobicity or whether molecule (or group or moiety) is hydrophobic are well known in the art and include empirically derived methods, as well as calculation methods. Exemplary methods are described in Zhu Chongqin et al. (2016) Characterizing hydrophobicity of amino acid side chains in a protein environment via measuring contact angle of a water nanodroplet on planar peptide network. Proc. Natl. Acad. Sci., 113(46) pgs. 12946-12951. As disclosed herein, exemplary hydrophobic moieties included, but are not limited to, Groups I, II, III, IV, V, VII, VIII, IX, XI, XII, XIII, XV and XVI of FIG. 1. Further exemplary hydrophobic moieties include those of FIG. 3 (e.g., Bn, Nap, PE, PP, iBu, 2Nap, Try, NE, MBn, BF, BT, Trp).

As used herein, an aptamer comprising a single type of 5-position modified pyrimidine or C-5 modified pyrimidine may be referred to as “single modified aptamers”, aptamers having a “single modified base”, aptamers having a “single base modification” or “single bases modified”, all of which may be used interchangeably. A library of aptamers or aptamer library may also use the same terminology. As used herein, “protein” is used synonymously with “peptide,” “polypeptide,” or “peptide fragment.” A “purified” polypeptide, protein, peptide, or peptide fragment is substantially free of cellular material or other contaminating proteins from the cell, tissue, or cell-free source from which the amino acid sequence is obtained, or substantially free from chemical precursors or other chemicals when chemically synthesized.

In certain embodiments, an aptamer comprises a first 5-position modified pyrimidine and a second 5-position modified pyrimidine, wherein the first 5-position modified pyrimidine comprises a tryosyl moiety at the 5-position of the first 5-position modified pyrimidine, and the second 5-position modified pyrimidine comprises a naphthyl moiety or benzyl moiety at the 5-position at the second 5-position modified pyrimidine. In a related embodiment the first 5-position modified pyrimidine is a uracil. In a related embodiment, the second 5-position modified pyrimidine is a cytosine. In a related embodiment, at least 10%, 15%, 20%, 25%, 30%, 35%, 40%, 45%, 50%, 55%, 60%, 65%, 70%, 75%, 80%, 85%, 90%, 95%, or 100% of the uracils of the aptamer are modified at the 5-position. In a related embodiment, at least 10%, 15%, 20%, 25%, 30%, 35%, 40%, 45%, 50%, 55%, 60%, 65%, 70%, 75%, 80%, 85%, 90%, 95%, or 100% of the cytosine of the aptamer are modified at the 5-position.

Those of ordinary skill in the art of nucleic acid hybridization will recognize that factors commonly used to impose or control stringency of hybridization include formamide concentration (or other chemical denaturant reagent), salt concentration (i.e., ionic strength), hybridization temperature, detergent concentration, pH and the presence or absence of chaotropes. Optimal stringency for a probe/target sequence combination is often found by the well-known technique of fixing several of the aforementioned stringency factors and then determining the effect of varying a single stringency factor. The same stringency factors can be modulated to thereby control the stringency of hybridization of a PNA to a nucleic acid, except that the hybridization of a PNA is fairly independent of ionic strength. Optimal stringency for an assay may be experimentally determined by examination of each stringency factor until the desired degree of discrimination is achieved.

As used herein, “Hybridization,” “hybridizing,” “binding” and like terms, in the context of nucleotide sequences, can be used interchangeably herein. The ability of two nucleotide sequences to hybridize with each other is based on the degree of complementarity of the two sequences, which in turn is based on the fraction of matched complementary nucleotide pairs. The more nucleotides in a given sequence that are complementary to another sequence, the more stringent the conditions can be for hybridization and the more specific will be the binding of the two sequences. Increased stringency is achieved by elevating the temperature, increasing the ratio of co-solvents, lowering the salt concentration, and the like. Hybridization of complementary Watson/Crick base pairs of probes on the microarray and of the target material is generally preferred, but non-Watson/Crick base pairing during hybridization can also occur.

Conventional hybridization solutions and processes for hybridization are described in J. Sambrook, Molecular Cloning: A Laboratory Manual, (supra), incorporated herein by reference. Conditions for hybridization typically include (1) high ionic strength solution, (2) at a controlled temperature, and (3) in the presence of carrier DNA and surfactants and chelators of divalent cations, all of which are known in the art.

As used herein, “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. As such, this term includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes deoxyribonucleic acid or DNA (including cDNA), ribonucleic acid or RNA and oligonucleotides, regardless of the source.

As used herein, “array” includes any one, two or three-dimensional arrangement of addressable regions bearing a particular chemical moiety or moieties (for example, biopolymers such peptide nucleic acid molecules, peptides or polynucleotide sequences) associated with that region, where the chemical moiety or moieties are immobilized on the surface in that region. By “immobilized” is meant that the moiety or moieties are stably associated with the substrate surface in the region, such that they do not separate from the region under conditions of using the array, e.g., hybridization and washing and stripping conditions. As is known in the art, the moiety or moieties may be covalently or non-covalently bound to the surface in the region. For example, each region may extend into a third dimension in the case where the substrate is porous while not having any substantial third dimension measurement (thickness) in the case where the substrate is non-porous. An array may contain more than ten, more than one hundred, more than one thousand more than ten thousand features, or even more than one hundred thousand features, in an area of less than 20 cm or even less than 10 cm. For example, features may have widths (that is, diameter, for a round spot) in the range of from about 10 μm to about 1.0 cm. In other embodiments each feature may have a width in the range of about 1.0 μm to about 1.0 mm, such as from about 5.0 μm to about 500 μm, and including from about 10 μm to about 200 μm. Non-round features may have area ranges equivalent to that of circular features with the foregoing width (diameter) ranges. A given feature is made up of chemical moieties, e.g., peptide nucleic acid molecules, peptides, nucleic acids, that bind to (e.g., hybridize to) the target molecule (e.g., target nucleic acid or aptamer), such that a given feature corresponds to a particular target.

In the case of an array, the “target” will be referenced as a moiety in a mobile phase (typically fluid), to be detected by probes (“target probes”) which are bound to the substrate at the various regions. However, either of the “target” or “target probes” may be the one which is to be detected by the other. In some embodiments, the target is an oligonucleotide or aptamer. In some embodiments, the probe is a peptide nucleic acid molecule, peptide, protein, oligonucleotide or aptamer.

The term “biological sample”, “sample”, and “test sample” are used interchangeably herein to refer to any material, biological fluid, tissue, or cell obtained or otherwise derived from an individual, and environmental, animal, or food sample. This includes blood (including whole blood, leukocytes, peripheral blood mononuclear cells, buffy coat, plasma, and serum), sputum, tears, mucus, nasal washes, nasal aspirate, breath, urine, semen, saliva, peritoneal washings, ascites, cystic fluid, meningeal fluid, amniotic fluid, glandular fluid, lymph fluid, nipple aspirate, bronchial aspirate (e.g., bronchoalveolar lavage), bronchial brushing, synovial fluid, joint aspirate, organ secretions, cells, a cellular extract, and cerebrospinal fluid. This also includes experimentally separated fractions of all of the preceding. For example, a blood sample can be fractionated into serum, plasma, or into fractions containing particular types of blood cells, such as red blood cells or white blood cells (leukocytes). In some embodiments, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas, and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual.

The phrase “oligonucleotide bound to a surface of a solid support” or “probe bound to a solid support” or a “target bound to a solid support” refers to a peptide nucleic acid molecules, oligonucleotide, aptamer, e.g., PNA (peptide nucleic acid), LNA (locked nucleic acid) or UNA (unlocked nucleic acid) molecule that is immobilized on a surface of a solid substrate, where the substrate can have a variety of configurations, e.g., a sheet, bead, particle, slide, wafer, web, fiber, tube, capillary, microfluidic channel or reservoir, or other structure. In certain embodiments, the collections of oligonucleotide or target elements employed herein are present on a surface of the same planar support, e.g., in the form of an array. It should be understood that the terms “probe” and “target” are relative terms and that a molecule considered as a probe in certain assays may function as a target in other assays. Immobilization of oligonucleotides on a substrate or surface can be accomplished by well-known techniques, commonly available in the literature. See for example A. C. Pease, et al., Proc. Nat. Acad. Sci, USA, 91:5022-5026 (1994); Z. Guo, et al., Nucleic Acids Res, 22, 5456-65 (1994); and M. Schena, et al., Science, 270, 467-70 (1995), each incorporated by reference herein.

The foregoing chemistry of the synthesis of polynucleotides is described in detail, for example, in Caruthers, Science 230: 281-285, 1985; Itakura et al., Ann. Rev. Biochem. 53: 323-356; Hunkapillar et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. Nos. 4,458,066, 4,500,707, 5,153,319, 5,869,643, EP 0294196, and elsewhere. The phosphoramidite and phosphite triester approaches are most broadly used, but other approaches include the phosphodiester approach, the phosphotriester approach and the H-phosphonate approach. The substrates are typically functionalized to bond to the first deposited monomer. Suitable techniques for functionalizing substrates with such linking moieties are described, for example, in Southern, E. M., Maskos, U. and Elder, J. K., Genomics, 13, 1007-1017, 1992. In the case of array fabrication, different monomers and activator may be deposited at different addresses on the substrate during any one cycle so that the different features of the completed array will have different desired biopolymer sequences. One or more intermediate further steps may be required in each cycle, such as the conventional oxidation, capping and washing steps in the case of in situ fabrication of polynucleotide arrays (again, these steps may be performed in flooding procedure).

Multiplex Assay

Multiplexed aptamer assays in solution-based target interaction and separation steps are described, e.g. in U.S. Pat. Nos. 7,855,054 and 7,964,356 and PCT Application PCT/US2013/044792. In one embodiment, a multiplex assay is described herein at Example 1.

In a multiplex assay format where multiple target proteins are being measured by multiple capture reagents, the natural variation in the abundance of the different target proteins can limit the ability of certain capture reagents to measure certain target proteins (e.g., high abundance target proteins may saturate the assay and prevent or reduce the ability of the assay to measure low abundance target proteins). To address this variation in the biological sample, the aptamer reagents may be separated into at least two different groups (Capture Reagents for DIL1 and Capture Reagents for DIL2), preferably three different groups (A3—Capture Reagents for DIL1; A2—Capture Reagents for DIL2 and A1—Capture Reagents for DIL3), based on the abundance of their respective protein target in the biological sample. Each of the capture reagent groups, A1, A2 and A3 each have a different set of aptamers, with the aptamers having specific affinity for a target protein. The biological sample is diluted into two (Dilution 1 or DIL1 and Dilution 2 or DIL2), preferably three, different dilution groups (Dilution 1 or DIL1; Dilution 2 or DIL2 and Dilution 3 or DIL3) to create separate test samples based on relative concentrations of the protein targets to be detected by their capture reagents. Thus, the biological sample is diluted into high, medium and low abundant target protein dilution groups, where the least abundant protein targets are measured in the least diluted group, and the most abundant protein targets are measured in the greatest diluted group. The capture reagents for their respective dilution groups are incubated together (e.g., the A3 set of aptamers are incubated with the test sample of Dilution 1 or DIL1; the A2 set of aptamers are incubated with the test sample of Dilution 2 or DIL2 and the A1 set of aptamers are incubated with the test sample of Dilution 3 or DIL3). The total number of aptamers for A1, A2 and A3 may be 4,000; 4,500; 5,000 or more aptamers.

FIG. 5 provides an example overview of the dilution sets for a biological sample, the corresponding capture reagent sets for their respective dilutions, and the general overview of the two-catch system (catch-1 and catch-2). Three different dilution groups may be created from a biological sample that includes a Z% dilution of the biological sample or DIL3, a Y% dilution of the biological sample or DIL2 and a X% dilution of the biological sample or DIL1, where Z is greater than Y, and Y is greater than X (or Z is a greater dilution than the Y dilution, and the Y dilution is a greater dilution than the X dilution). Each dilution has its own set of corresponding capture reagents (A3 for DIL1, A2 for DIL2 and A1 for DIL3) that bind to a specific set of proteins.

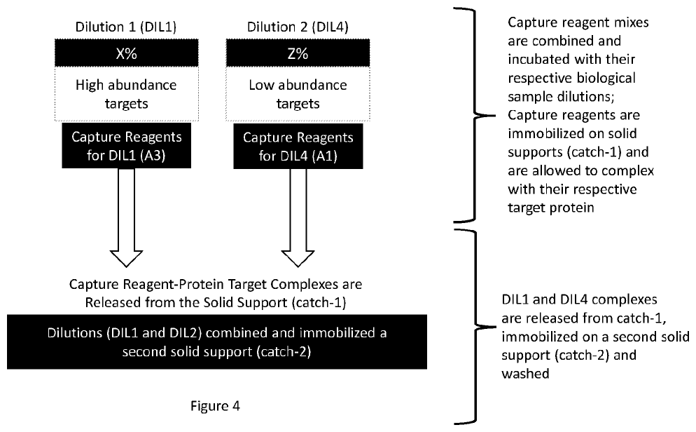

FIG. 4 provides an example overview of the dilution sets for a biological sample, the corresponding capture reagent sets for their respective dilutions, and the general overview of the two-catch system (catch-1 and catch-2). Two different dilution groups may be created from a biological sample that includes a Z% dilution of the biological sample or DIL4 and an X% dilution of the biological sample or DIL1, where Z is greater than X (or Z is a greater dilution than the X dilution). Each dilution has its own set of corresponding capture reagents (A3 for DIL1 and A1 for DIL4) that bind to a specific set of proteins.

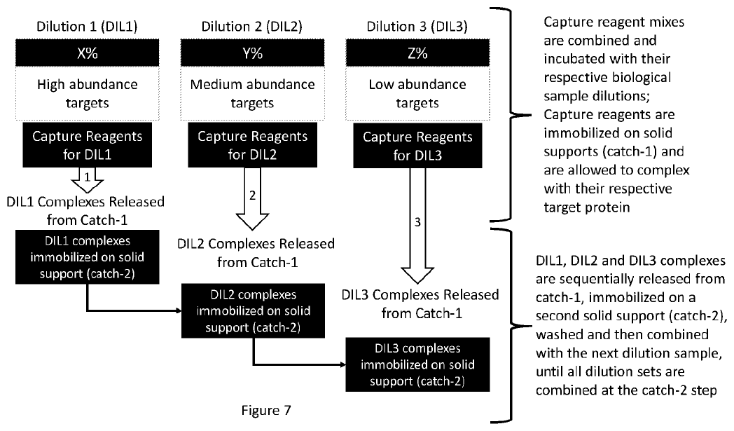

FIG. 7 provides an example overview of the dilution sets for a biological sample, the corresponding capture reagent sets for their respective dilutions, and the general overview of the sequential two-catch system (catch-1 and catch-2). Three different dilution groups may be created from a biological sample that includes a Z% dilution of the biological sample or DIL3, a Y% dilution of the biological sample or DIL2 and a X% dilution of the biological sample or DIL1, where Z is greater than Y, and Y is greater than X (or Z is a greater dilution than the Y dilution, and the Y dilution is a greater dilution than the X dilution). Each dilution has its own set of corresponding capture reagents (A3 for DIL1, A2 for DIL2 and A1 for DIL3) that bind to a specific set of proteins.

The present disclosure describes improved methods to perform aptamer- and photoaptamer-based multiplexed assays for the quantification of one or more target molecule(s) that may be present in a test sample wherein the aptamer (or photoaptamer) can be separated from the aptamer-target affinity complex (or photoaptamer-target covalent complex) for final detection using any suitable nucleic acid detection method in as much as the materials and methods described herein can be used to improve overall assay performance. Photoaptamers are aptamers that comprise photoreactive functional groups that enable the aptamers to covalently bind or “photocros slink” their target molecules.

The improved aptamer- and photoaptamer-based multiplexed assays described herein can be performed with aptamers and photoaptamers, including but not limited to those aptamers and photoaptamers described in the publications listed in Table 1.

TABLE 1

| | Filing | | WO Publication |
|---|---|---|---|
| Application No. | Date | Title | No. |
| PCT/US2016/050908 | Sep. 9, | Methods for Developing | WO/2017/ |
| | 2016 | Personalized Drug | 044715 |
| | | Treatment Plans and | |
| | | Targeted Drug | |
| | | Development Based on | |
| | | Proteomic Profiles | |
| PCT/US2016/16712 | Feb. 5, | Nucleic Acid | WO/2016/ |
| | 2016 | Compounds for Binding | 130414 |
| | | Growth Differentiation | |
| | | Factor 8 | |
| PCT/US2015/62155 | Nov. 23, | Nucleic Acid | WO/2016/ |
| | 2015 | Compounds for Binding | 085860 |
| | | Growth Differentiation | |
| | | Factor 11 | |
| PCT/US2015/33355 | May 29, | Nucleic Acid | WO/2015/ |
| | 2015 | Compounds for Binding | 184372 |
| | | to Complement | |
| | | Component 3 Protein | |
| PCT/US2014/054561 | Sep. 8, | PDGF and VEGF | WO/2015/ |
| | 2014 | Aptamers Having | 035305 |
| | | Improved Stability and | |
| | | Their Use in Treating | |
| | | PDGF and VEGF | |
| | | Mediated Diseases and | |
| | | Disorders | |
| PCT/US2014/024669 | Mar. 12, | Aptamers That Bind to | WO/2014/ |
| | 2014 | Il-6 and Their Use in | 159669 |
| | | Treating or Diagnosing | |
| | | Il-6 Mediated | |
| | | Conditions | |
| PCT/US2013/034493 | Mar. 28, | Aptamers to PDGF and | WO/2013/ |
| | 2013 | VEGF and Their Use in | 149086 |
| | | Treating PDGF and | |
| | | VEGF Mediated | |
| | | Conditions | |
| PCT/US2012/72094 | Dec. 28, | Aptamers and | WO/2013/ |
| | 2012 | Diagnostic Methods for | 102096 |
| | | Detecting the EGF | |
| | | Receptor | |
| PCT/US2012/072101 | Dec. 28, | Aptamers and | WO/2013/ |
| | 2012 | Diagnostic Methods for | 102101 |
| | | Detecting the EGF | |
| | | Receptor | |
| PCT/US2012/028632 | Mar. 9, | Aptamers for | WO/2012/ |
| | 2012 | Clostridium Difficile | 122540 |
| | | Diagnostics | |
| PCT/US2011/032017 | Apr. 12, | Aptamers to β-NGF and | WO/2011/ |
| | 2011 | Their Use in Treating β- | 130195 |
| | | NGF Mediated Diseases | |
| | | and Disorders | |
| PCT/US2011/027064 | Mar. 3, | Aptamers to 4-1BB and | WO/2011/ |
| | 2011 | Their Use in Treating | 109642 |
| | | Diseases and Disorders | |

Historically, two unanticipated limitations emerged from performing single- and multi-plex aptamer based assays, including multiplexed proteomic aptamer affinity assays. First, aptamer/aptamer interactions were identified as a primary source of assay background and a potential limitation to multiplex capacity. Second, sample matrices (primarily serum and plasma) were found to inhibit the immobilization of biotinylated aptamers on streptavidin-substituted matrices.

An improvement in the assay, as described in Gold et al. (PLoS One (2010) £12):el5005), comprised the use of organic solvents in some of the wash buffers of the Catch-2 step to diminish the dielectric constant of the medium. Addition of these wash buffers effectively accented the like-charge repulsion of adjacent phosphodiester backbones of the aptamers, thus promoting dissociation of background-causing interacting aptamers.

Another improvement in the process involves the addition of organic solvents to some of wash buffers used in the Catch-2 step of the assay, it also counters the tendency of aptamers to interact, and thus diminishes background and increases multiplex capacity. However, its primary advantage is to counteract the matrix-dependent inhibition of biotinylated aptamer adsorption to streptavidin matrices. Such inhibition is easily detectable even at 5% v/v plasma or serum, and limits working assay concentrations to 5-10% plasma or serum concentrations. This limitation in turn limits assay sensitivity.

Yet another improvement to the multiplexed assay comprises pre-immobilization of the tagged aptamers on the solid support matrices prior to incubation (termed “Catch-0”) with the test solution. Incubation with the test solution is then carried out with bound aptamers, in the processing vessels themselves. As described herein for purposes of illustration only, biotinylated aptamers were pre-immobilized on streptavidin bead matrices, and incubation with test solution carried out with the bead-bound aptamers. This pre-immobilization step enables immobilization under conditions where aptamers have diminished tendency to interact and also enables very stringent washes (with base and with chaotropic salts) prior to incubation, disrupting interacting aptamers and removing all aptamers not bound through the very robust biotin-streptavidin interaction. This reduces the number of aptamer “clumps” traversing the assay-clumps that have at some detectable frequency retained the biotin moiety or become biotinylated in the assay. It is worth noting that irradiation cleaves most, but not all photocleavable biotin moieties from aptamers, while some aptamers become biotinylated via the NHS-biotin treatment intended to “tag” proteins. Biotinylated aptamer that is captured at the Catch-2 step creates background by interacting with bulk photocleaved aptamer, which is then released upon elution. It should also be noted that a pre-immobilized format will likely support very high multiplex capacities as aptamer panels may be immobilized separately then combined in bead-bound form, thus bypassing conditions in which aptamers may interact and clump.

Thus, pre-immobilization bypasses the need for aptamer adsorption in the presence of analyte solution, thus ensuring quantitative immobilization even when assaying inhibitory concentrations of analyte solutions. This enables the use of much higher concentrations, up to and including at least 40% v/v plasma or serum, rather than the 10% top concentration of the process as previously described (Gold et al. (Dec. 2010) PLoS One 5(12):el5005) or the 5% top concentration used in more recent editions of the process thereby increasing sensitivity roughly 4- to 8-fold, as well as, increasing the overall robustness of the assay.

Another improvement to the overall process comprises the use of a chaotropic salt at about a neutral pH for elution during the Catch-2 step as described in detail below. Prior methods comprised the use of sodium chloride at high pH (10), which disrupts DNA hybridization and aptamer/aptamer interaction as well as protein/aptamer interaction. As noted above, DNA hybridization and aptamer/aptamer interactions contribute to assay background. Chaotropic salts, including but not limited to sodium perchlorate, lithium chloride, sodium chloride and magnesium chloride at neutral pH, support DNA hybridization and aptamer/aptamer interactions, while disrupting aptamer/protein interactions. The net result is significantly diminished (about 10-fold) background, with a concomitant rise in assay sensitivity.

As used herein “Catch-1” refers to the partitioning of an aptamer-target affinity complex or aptamer-target covalent complex. The purpose of Catch-1 is to remove substantially all of the components in the test sample that are not associated with the aptamer. Removing the majority of such components will generally improve target tagging efficiency by removing non-target molecules from the target tagging step used for Catch-2 capture and may lead to lower assay background. In one embodiment, a tag is attached to the aptamer either before the assay, during preparation of the assay, or during the assay by appending the tag to the aptamer. In one embodiment, the tag is a releasable tag. In one embodiment, the releasable tag comprises a cleavable linker and a tag. As described above, tagged aptamer can be captured on a solid support where the solid support comprises a capture element appropriate for the tag. The solid support can then be washed as described herein prior to equilibration with the test sample to remove any unwanted materials (Catch-0).

As used herein “Catch-2” refers to the partitioning of an aptamer-target affinity complex or aptamer-target covalent complex based on the capture of the target molecule. The purpose of the Catch-2 step is to remove free, or uncomplexed, aptamer from the test sample prior to detection and optional quantification. Removing free aptamer from the sample allows for the detection of the aptamer-target affinity or aptamer-target covalent complexes by any suitable nucleic acid detection technique. When using Q-PCR for detection and optional quantification, the removal of free aptamer is needed for accurate detection and quantification of the target molecule.

In one embodiment, the target molecule is a protein or peptide and free aptamer is partitioned from the aptamer-target affinity (or covalent) complex (and the rest of the test sample) using reagents that can be incorporated into proteins (and peptides) and complexes that include proteins (or peptides), such as, for example, an aptamer-target affinity (or covalent) complex. The tagged protein (or peptide) and aptamer-target affinity (or covalent) complex can be immobilized on a solid support, enabling partitioning of the protein (or peptide) and the aptamer-target affinity (or covalent) complex from free aptamer. Such tagging can include, for example, a biotin moiety that can be incorporated into the protein or peptide.

In one embodiment, a Catch-2 tag is attached to the protein (or peptide) either before the assay, during preparation of the assay, or during the assay by chemically attaching the tag to the targets. In one embodiment the Catch-2 tag is a releasable tag. In one embodiment, the releasable tag comprises a cleavable linker and a tag. It is generally not necessary, however, to release the protein (or peptide) from the Catch-2 solid support. As described above, tagged targets can be captured on a second solid support where the solid support comprises a capture element appropriate for the target tag. The solid support is then washed with various buffered solutions including buffered solutions comprising organic solvents and buffered solutions comprising salts and/or detergents containing salts and/or detergents.

After washing the second solid support, the aptamer-target affinity complexes are then subject to a dissociation step in which the complexes are disrupted to yield free aptamer while the target molecules generally remain bound to the solid support through the binding interaction of the capture element and target capture tag. The aptamer can be released from the aptamer-target affinity complex by any method that disrupts the structure of either the aptamer or the target. This may be achieved though washing of the support bound aptamer-target affinity complexes in high salt buffer which dissociates the non-covalently bound aptamer-target complexes. Eluted free aptamers are collected and detected. In another embodiment, high or low pH is used to disrupt the aptamer-target affinity complexes. In another embodiment high temperature is used to dissociate aptamer-target affinity complexes. In another embodiment, a combination of any of the above methods may be used. In another embodiment, proteolytic digestion of the protein moiety of the aptamer-target affinity complex is used to release the aptamer component.

In the case of aptamer-target covalent complexes, release of the aptamer for subsequent quantification is accomplished using a cleavable linker in the aptamer construct. In another embodiment, a cleavable linker in the target tag will result in the release of the aptamer-target covalent complex.

By way of example, the proteomic affinity assay (multiplex assay) may be practiced as follows:

Catch-0: 133 7.5% streptavidin-agarose slurry in 1×SB17,Tw (40 mM HEPES, 102 mM NaCl, 1 mM EDTA, 5 mM MgCl2, 5 mM KCl, 0.05% Tween-20) was added to wells of the filter plate (0.45 μmMillipore HV plates (Durapore cat# MAHVN4550)). The appropriate 1.1× aptamer mix (all aptamers contain a Cy3 fluorophore and a photocleavable biotin moiety on the 5′ end) was thawed followed by vortexing. The 1.1× aptamer mix was then boiled for 10 min, vortexed for 30 s and allowed to cool to 20° C. in a water bath for 20 min. The liquid in the filter plates containing the streptavidin agarose slurry was then removed by centrifugation (1000×g for 1 minute). 100 μL aptamer mix was added to the wells of the filter plate (robotically). The mixture was incubated at 25° C. for 20 min on a shaker set at 850 rpm, protected from light.

Catch-0 washes: Subsequent to the 20 min incubation the solution was removed via vacuum filtration. 190 1× CAPS aptamer prewash buffer (50 mM CAPS, 1 mM EDTA, 0.05% Tw-20, pH 11.0) was added and the mixture was incubated for 1 minutes while shaking. The CAPS wash solution was then removed via vacuum filtration. The CAPS wash was then repeated one time. 190 μL 1× SX17-Tween was added and the mixture was incubated for 1 min while shaking. The 1× SB17-Tween was then removed via vacuum filtration. An additional 190 μL 1× SX17-Tw was added and the mixture was incubated for 1 min while shaking. The 1× SB17-Tw was then removed by centrifugation (1 min at 1000×g). Following removal of the 1× SB17,Tw, 150 pt. Catch-0 storage buffer (150 mM NaCl, 40 mM HEPES, 1 mM EDTA, 0.02% sodium azide, 0.05% Tween-20) was added and the filter plate was carefully sealed at the plate perimeter only and stored at 4° C. in the dark until use.

Sample Preparation: Seventy-five (75) microliters of 40% sample diluent were plated out in a 40% sample plate (Final 40% sample contains: 20 μM Z-block, 1 mM benzamidine, 1 mM EGTA, 40 mM HEPES, 5 mM MgCl2, 5 mM KCl, 1% Tween-20). One hundred ninety-five (195) microliters of 1× SB17-Tw were plated out in a 1% sample plate. Ninety (90) microliters of 1× SB17-Tw were plated out in a 1 to 10 dilution plate. One hundred thirty-three (133) microliters·1× SB17-Tw were plated out in a 0.005% sample plate. Samples were thawed for 10 min on the Rack Thawing Station in a 25° C. incubator, then vortexed and spun at 1000×g for 1 minute. The caps were removed from the tubes. The samples were mixed (5 times with 50 μL) and 50 μL 100% sample was transferred to the 40% sample plate containing the sample diluents. The 40% sample was then mixed on the sample plate by pipetting up and down (110 μL, 10 times). Five (5) μL of 40% sample was then transferred to the 1% sample plate containing 1× SB17-Tw. Again this sample was mixed by pipetting up and down (120 μL, 10 times). After mixing, 10 μL of the 1% sample was transferred to the 1 to 10 dilution plate containing 1× SB17-Tw, which was mixed by pipetting up and down (75 μL, 10 times). Seven (7) microliters of the 0.1% sample from the 1 to 10 dilution plate was transferred into the 0.005% sample plate containing 1× SB17-Tw and mixed by pipetting up and down (110 μL, 10 times).

Plate Preparation before Incubation: The Catch-0 storage solution was removed from the filter plates via vacuum filtration. One hundred ninety (190) microliters of 1× SB17-Tw was then added followed by removal from the filter plates via vacuum filtration. An additional 190 μL 1× SB17-Tw was then added to the filter plates.

Incubation: The 1× SB17-Tw buffer was removed from the filter plates by centrifugation (1 min. at 1000x g). One hundred (100) microliters of the appropriate sample dilution was added to the filter plates (three filter plates, one for each sample dilution 40% or 20%, 1%, or 0.005%). The filter plates were carefully sealed at the plate perimeter only, avoiding pressurizing the wells. Pressure will cause leakage during incubation. The plates were then incubated for 3.5 hours at 28° C. on the thermoshaker set at 850 rpm, protected from light. Filter Plate Processing: After incubation, the filter plates were placed onto vacuum manifolds and the sample was removed by vacuum filtration. One hundred ninety (190) microliters, biotin wash (100 μM biotin in 1× SB17-Tw) was added and the liquid was removed by vacuum filtration. The sample was then washed 5× with 190 μL 1× SB17-Tw (vacuum filtration). One hundred (100) microliters of 1 mM NHS-biotin in 1× SB17-Tw (freshly prepared) was added and the filter plates were blotted on an absorbent pad and the mixture was incubated for 5 minutes with shaking. The liquid was removed by vacuum filtration. One hundred and twenty five (125) microliters 20 mM glycine in 1× SB17-Tw was added and the liquid was removed by vacuum filtration. Again 125 μL 20 mM glycine in 1× SB17-Tw was added and the liquid removed by vacuum filtration.

Subsequently the samples were washed 6× with 190 μL 1× SB17-Tw, with the liquid being removed by vacuum filtration. Eighty five (85) microliters of photocleavage buffer (2 μM Z-block in 1× SB17-Tw) was then added to each of the filter plates.

Photocleavage: The filter plates were blotted on absorbent pads and were irradiated for 6 min with a BlackRay UV lamp with shaking (800 rpm, 25° C.). The plates were rotated 180 degrees and irradiated for an additional 6 min. under the BlackRay light source. The 40% filter plate was placed onto an empty 96-well plate. The 1% filter plate was stacked on top of the 40% filter plate and the 0.005% filter plate was stacked on top of the 1% filter plate. The assembly of plated were spun for 1 min at 1000x g. The 96-well plate with eluted sample was placed onto the robot deck. Sixty (60) percent glycerol in 1× SB17-Tw from the 37° C. incubator was placed onto the robotic deck.

Catch-2: During assay setup 50 μL of 10 mg/mL MyOne SA beads (500 μg) was added to an ABgene Omni-tube 96-well plate for Catch-2 and placed in the Cytomat. The Catch-2 96-well bead plate was suspended for 90 s., placed on magnet block for 60 s. and the supernatant was removed. At the same time, or sequentially, the Catch-1 eluate from each dilution group was transferred to the Catch-2 bead plate and incubated on a Peltier thermoshaker (1350 rpm, 5 min, 25° C.). The plate was transferred to a 25° C. magnet for 2 minutes and the supernatant was removed. Next 75 μL 1× SB17-Tw was added and the sample and incubated on a Peltier shaker at 1350 rpm for 1 minute at 37° C. Then 75 μL 60% glycerol in 1× SB17-Tw (heated to 37° C.) was added and the sample was again incubated on the Peltier Shaker at 1350 rpm for 1 minute at 37° C. The plate was transferred to a magnet heated to 37° C. and incubated for 2 min. followed by the removal of the supernatant. This 37° C. 1× SB17-Tw and glycerol wash cycle was repeated two more times. The sample was then washed to remove residual glycerol with 150 μL 1× SB17-Tw on a Peltier shaker (1350 rpm, 1 minute, 25° C.), followed by 1 minute on a 25° C. magnetic block. The supernatant was removed and 150 μL 1× SB17-Tw substituted with 0.5 M NaCl was added and incubated at 1350 rpm for 1 minute (25° C.) followed by 1 minute on a 25° C. magnetic block. The supernatant was removed and 75 μL perchlorate elution buffer (1.8 M NaClC-4, 40 mM PIPES, 1 mM EDTA, 0.05% Triton X-100, 1× Hybridization controls, pH=6.8) was added followed by a 10 minute incubation on a Peltier shaker (25° C., 1350 rpm). Afterwards the plate was transferred to a magnetic separator and incubated for 90 s, and the supernatant was recovered.

Hybridization: Twenty (20) microliters eluted sample was added robotically to an empty the 96-well plate. Five (5) microliters 10× Agilent blocking buffer containing a second set of hybridization controls were robotically added to the eluted samples. Then 25 μL 2× Agilent HiRPM hybridization buffer was added manually to the wells. Forty (40) microliters of hybridization mix was loaded onto the Agilent gasket slide. The Agilent 8 by 15 k array was added onto gasket slide and the sandwich was tightened with a clamp. The sandwich was then incubated rotating (20 rpm) for 19 hours at 55° C.

Post-Hybridization Washing: Post hybridization slide processing was performed on a Little Dipper Processor (SciGene, Cat# 1080-40-1). Approximately 750 mL wash buffer 1 (Oligo aCGH/ChIP-on-chip Wash Buffer 1, Agilent Technologies) was placed into one glass staining dish. Approximately 750 mL wash buffer 1 (Oligo aCGH/ChIP-on-chip Wash Buffer 1, Agilent Technologies) was placed into Bath #1 of the Little Dipper Processor. Approximately 750 mL wash buffer 2 (Oligo aCGH/ChIP-on-chip Wash Buffer 1, Agilent Technologies) heated to 37° C. was placed into Bath #2 of the Little Dipper Processor. The magnetic stir speed for both bath were set to 5. The temperature controller for Bath #1 was not turned on, while the temperature controller for Bath #2 was set to 37° C. Up to twelve slide/gasket assemblies were sequentially disassembled into the first staining dish containing Wash Buffer 1 and the slides were placed into a slide rack while still submerged in Wash Buffer 1. Once all slide/gaskets assemblies were disassembled, the slide rack was quickly transferred into Bath #1 of the Little Dipper Processor and the automated wash protocol was started. The Little Dipper Processor incubated the slides for 300 s. in Bath #1 at a speed of 250 followed by a transfer to the 37° C. Bath #2 containing the Agilent Wash 2 (Oligo aCGH/ChIP-on-chip Wash Buffer 2, Agilent Technologies) and incubated for 300 s. at speed 100. Afterwards the Little Dipper Processor transferred the slide rack to the built-in centrifuge, where the slides were spun for 300 s at speed 690.

Microarray Imaging: The microarray slides were imaged with a microarray scanner (Agilent G2565CA Microarray Scanner System, Agilent Technologies) in the Cy3-channel at 5 μm resolution at 100% PMT setting and the XRD option enabled at 0.05. The resulting tiff images were processed using Agilent feature extraction software version 10.7.3.1 with the GE1_107_Sep09 protocol.

As used herein, a “releasable” or “cleavable” element, moiety, or linker refers to a molecular structure that can be broken to produce two separate components. A releasable (or cleavable) element may comprise a single molecule in which a chemical bond can be broken (referred to herein as an “inline cleavable linker”), or it may comprise two or more molecules in which a non-covalent interaction can be broken or disrupted (referred to herein as a “hybridization linker”).

In some embodiments, it is necessary to spatially separate certain functional groups from others in order to prevent interference with the individual functionalities. For example, the presence of a label, which absorbs certain wavelengths of light, proximate to a photocleavable group can interfere with the efficiency of photocleavage. It is therefore desirable to separate such groups with a non-interfering moiety that provides sufficient spatial separation to recover full activity of photocleavage, for example. In some embodiments, a “spacing linker” has been introduced into an aptamer with both a label and photocleavage functionality.

“Solid support” refers to any substrate having a surface to which molecules may be attached, directly or indirectly, through either covalent or non-covalent bonds. The solid support may include any substrate material that is capable of providing physical support for the capture elements or probes that are attached to the surface. The material is generally capable of enduring conditions related to the attachment of the capture elements or probes to the surface and any subsequent treatment, handling, or processing encountered during the performance of an assay. The materials may be naturally occurring, synthetic, or a modification of a naturally occurring material. Suitable solid support materials may include silicon, a silicon wafer chip, graphite, mirrored surfaces, laminates, membranes, ceramics, plastics (including polymers such as, e.g., poly(vinyl chloride), cyclo-olefin copolymers, agarose gels or beads, polyacrylamide, polyacrylate, polyethylene, polypropylene, poly(4-methylbutene), polystyrene, polymethacrylate, poly(ethylene terephthalate), polytetrafluoroethylene (PTFE or Teflon®), nylon, poly(vinyl butyrate)), germanium, gallium arsenide, gold, silver, Langmuir Blodgett films, a flow through chip, etc., either used by themselves or in conjunction with other materials. Additional rigid materials may be considered, such as glass, which includes silica and further includes, for example, glass that is available as Bioglass. Other materials that may be employed include porous materials, such as, for example, controlled pore glass beads, crosslinked beaded Sepharose® or agarose resins, or copolymers of crosslinked bis-acrylamide and azalactone. Other beads include nanoparticles, polymer beads, solid core beads, paramagnetic beads, or microbeads. Any other materials known in the art that are capable of having one or more functional groups, such as any of an amino, carboxyl, thiol, or hydroxyl functional group, for example, incorporated on its surface, are also contemplated.

The material used for a solid support may take any of a variety of configurations ranging from simple to complex. The solid support can have any one of a number of shapes, including a strip, plate, disk, rod, particle, bead, tube, well (microtiter), and the like. The solid support may be porous or non-porous, magnetic, paramagnetic, or non-magnetic, polydisperse or monodisperse, hydrophilic or hydrophobic. The solid support may also be in the form of a gel or slurry of closely-packed (as in a column matrix) or loosely-packed particles.

In one embodiment, the solid support with attached capture element is used to capture tagged aptamer-target affinity complexes or aptamer-target covalent complexes from a test mixture. In one particular example, when the tag is a biotin moiety, the solid support could be a streptavidin-coated bead or resin such as Dynabeads M-280 Streptavidin, Dynabeads MyOne Streptavidin, Dynabeads M-270 Streptavidin (Invitrogen), Streptavidin Agarose Resin (Pierce), Streptavidin Ultralink Resin, MagnaBind Streptavidin Beads (ThermoFisher Scientific), BioMag Streptavidin, ProMag Streptavidin, Silica Streptavidin (Bangs Laboratories), Streptavidin Sepharose High Performance (GE Healthcare),

Streptavidin Polystyrene Microspheres (Microspheres-Nanospheres), Streptavidin Coated Polystyrene Particles (Spherotech), or any other streptavidin coated bead or resin commonly used by one skilled in the art to capture biotin-tagged molecules.

As has been described above, one object of the instant invention is to convert a protein signal into an aptamer signal. As a result the quantity of aptamers collected/detected is indicative of, and may be directly proportional to, the quantity of target molecules bound and to the quantity of target molecules in the sample. A number of detection schemes can be employed without eluting the aptamer-target affinity or aptamer-target covalent complex from the second solid support after Catch-2 partitioning. In addition to the following embodiments of detection methods, other detection methods will be known to one skilled in the art.

Many detection methods require an explicit label to be incorporated into the aptamer prior to detection. In these embodiments, labels, such as, for example, fluorescent or chemiluminescent dyes can be incorporated into aptamers either during or post synthesis using standard techniques for nucleic acid synthesis. Radioactive labels can be incorporated either during synthesis or post synthesis using standard enzyme reactions with the appropriate reagents. Labeling can also occur after the Catch-2 partitioning and elution by using suitable enzymatic techniques. For example, using a primer with the above mentioned labels, PCR will incorporate labels into the amplification product of the eluted aptamers. When using a gel technique for quantification, different size mass labels can be incorporated using PCR as well. These mass labels can also incorporate different fluorescent or chemiluminescent dyes for additional multiplexing capacity. Labels may be added indirectly to aptamers by using a specific tag incorporated into the aptamer, either during synthesis or post synthetically, and then adding a probe that associates with the tag and carries the label. The labels include those described above as well as enzymes used in standard assays for colorimetric readouts, for example. These enzymes work in combination with enzyme substrates and include enzymes such as, for example, horseradish peroxidase (HRP) and alkaline phosphatase (AP). Labels may also include materials or compounds that are electrochemical functional groups for electrochemical detection.

For example, the aptamer may be labeled, as described above, with a radioactive isotope such as 32 P prior to contacting the test sample. Employing any one of the four basic assays, and variations thereof as discussed above, aptamer detection may be simply accomplished by quantifying the radioactivity on the second solid support at the end of the assay. The counts of radioactivity will be directly proportional to the amount of target in the original test sample. Similarly, labeling an aptamer with a fluorescent dye, as described above, before contacting the test sample allows for a simple fluorescent readout directly on the second solid support. A chemiluminescent label or a quantum dot can be similarly employed for direct readout from the second solid support, requiring no aptamer elution.

By eluting the aptamer or releasing photoaptamer-target covalent complex from the second solid support additional detection schemes can be employed in addition to those described above. For example, the released aptamer, photoaptamer or photoaptamer-target covalent complex can be run on a PAGE gel and detected and optionally quantified with a nucleic acid stain, such as SYBR Gold. Alternatively, the released aptamer, photoaptamer or photoaptamer covalent complex can be detected and quantified using capillary gel electrophoresis (CGE) using a fluorescent label incorporated in the aptamer as described above. Another detection scheme employs quantitative PCR to detect and quantify the eluted aptamer using SYBR Green, for example. Alternatively, the Invader® DNA assay may be employed to detect and quantify the eluted aptamer. Another alternative detection scheme employs next generation sequencing.

In another embodiment, the amount or concentration of the aptamer-target affinity complex (or aptamer-target covalent complex) is determined using a “molecular beacon” during a replicative process (see, e.g., Tyagi et ah, Nat. Biotech. J_6:49 53, 1998; U.S. Pat. No. 5,925,517). A molecular beacon is a specific nucleic acid probe that folds into a hairpin loop and contains a fluorophore on one end and a quencher on the other end of the hairpin structure such that little or no signal is generated by the fluorophore when the hairpin is formed. The loop sequence is specific for a target polynucleotide sequence and, upon hybridizing to the aptamer sequence the hairpin unfolds and thereby generates a fluorescent signal.

For multiplexed detection of a small number of aptamers still bound to the second solid support, fluorescent dyes with different excitation/emission spectra can be employed to detect and quantify two, or three, or five, or up to ten individual aptamers.

Similarly different sized quantum dots can be employed for multiplexed readouts. The quantum dots can be introduced after partitioning free aptamer from the second solid support. By using aptamer specific hybridization sequences attached to unique quantum dots multiplexed readings for 2, 3, 5, and up to 10 aptamers can be performed. Labeling different aptamers with different radioactive isotopes that can be individually detected, such as 32 P, 3 H, 113JC, and 3 JSJS, can also be used for limited multiplex readouts.

For multiplexed detection of aptamers released from the Catch-2 second solid support, a single fluorescent dye, incorporated into each aptamer as described above, can be used with a quantification method that allows for the identification of the aptamer sequence along with quantification of the aptamer level. Methods include but are not limited to DNA chip hybridization, micro-bead hybridization, next generation sequencing and CGE analysis.

In one embodiment, a standard DNA hybridization array, or chip, is used to hybridize each aptamer or photoaptamer to a unique or series of unique probes immobilized on a slide or chip such as Agilent arrays, Illumina BeadChip Arrays, NimbleGen arrays or custom printed arrays. Each unique probe is complementary to a sequence on the aptamer. The complementary sequence may be a unique hybridization tag incorporated in the aptamer, or a portion of the aptamer sequence, or the entire aptamer sequence. The aptamers released from the Catch-2 solid support are added to an appropriate hybridization buffer and processed using standard hybridization methods. For example, the aptamer solution is incubated for 12 hours with a DNA hybridization array at about 60° C. to ensure stringency of hybridization. The arrays are washed and then scanned in a fluorescent slide scanner, producing an image of the aptamer hybridization intensity on each feature of the array. Image segmentation and quantification is accomplished using image processing software, such as ArrayVision. In one embodiment, multiplexed aptamer assays can be detected using up to 25 aptamers, up to 50 aptamers, up to 100 aptamers, up to 200 aptamers, up to 500 aptamers, up to 1000 aptamers, and up to 10,000 aptamers.

In one embodiment, addressable micro-beads having unique DNA probes complementary to the aptamers as described above are used for hybridization. The micro-beads may be addressable with unique fluorescent dyes, such as Luminex beads technology, or use bar code labels as in the Illumina VeraCode technology, or laser powered transponders. In one embodiment, the aptamers released from the Catch-2 solid support are added to an appropriate hybridization buffer and processed using standard micro-bead hybridization methods. For example, the aptamer solution is incubated for two hours with a set of micro-beads at about 60° C. to ensure stringency of hybridization. The solutions are then processed on a Luminex instrument which counts the individual bead types and quantifies the aptamer fluorescent signal. In another embodiment, the VeraCode beads are contacted with the aptamer solution and hybridized for two hours at about 60° C. and then deposited on a gridded surface and scanned using a slide scanner for identification and fluorescence quantification. In another embodiment, the transponder micro-beads are incubated with the aptamer sample at about 60° C. and then quantified using an appropriate device for the transponder micro-beads. In one embodiment, multiplex aptamer assays can be detected by hybridization to micro-beads using up to 25 aptamers, up to 50 aptamers, up to 100 aptamers, up to 200 aptamers, and up to 500 aptamers.

The sample containing the eluted aptamers can be processed to incorporate unique mass tags along with fluorescent labels as described above. The mass labeled aptamers are then injected into a CGE instrument, essentially a DNA sequencer, and the aptamers are identified by their unique masses and quantified using fluorescence from the dye incorporated during the labeling reaction. One exemplary example of this technique has been developed by Althea Technologies.

In many of the methods described above, the solution of aptamers can be amplified and optionally tagged before quantification. Standard PCR amplification can be used with the solution of aptamers eluted from the Catch-2 solid support. Such amplification can be used prior to DNA array hybridization, micro-bead hybridization, and CGE readout.

In another embodiment, the aptamer-target affinity complex (or aptamer-target covalent complex) is detected and/or quantified using Q-PCR. As used herein, “Q-PCR” refers to a PCR reaction performed in such a way and under such controlled conditions that the results of the assay are quantitative, that is, the assay is capable of quantifying the amount or concentration of aptamer present in the test sample.

In one embodiment, the amount or concentration of the aptamer-target affinity complex (or aptamer-target covalent complex) in the test sample is determined using TaqMan® PCR. This technique generally relies on the 5′-3′ exonuclease activity of the oligonucleotide replicating enzyme to generate a signal from a targeted sequence. A TaqMan probe is selected based upon the sequence of the aptamer to be quantified and generally includes a 5′-end fluorophore, such as 6-carboxyfluorescein, for example, and a 3′-end quencher, such as, for example, a 6-carboxytetramethylfluorescein, to generate signal as the aptamer sequence is amplified using polymerase chain reaction (PCR). As the polymerase copies the aptamer sequence, the exonuclease activity frees the fluorophore from the probe, which is annealed downstream from the PCR primers, thereby generating signal. The signal increases as replicative product is produced. The amount of PCR product depends upon both the number of replicative cycles performed as well as the starting concentration of the aptamer.

In another embodiment, the amount or concentration of an aptamer-target affinity complex (or aptamer-target covalent complex) is determined using an intercalating fluorescent dye during the replicative process. The intercalating dye, such as, for example, SYBR® green, generates a large fluorescent signal in the presence of double-stranded DNA as compared to the fluorescent signal generated in the presence of single-stranded DNA. As the double-stranded DNA product is formed during PCR, the signal produced by the dye increases. The magnitude of the signal produced is dependent upon both the number of PCR cycles and the starting concentration of the aptamer.

In another embodiment, the aptamer-target affinity complex (or aptamer-target covalent complex) is detected and/or quantified using mass spectrometry. Unique mass tags can be introduced using enzymatic techniques described above. For mass spectroscopy readout, no detection label is required, rather the mass itself is used to both identify and, using techniques commonly used by those skilled in the art, quantified based on the location and area under the mass peaks generated during the mass spectroscopy analysis. An example using mass spectroscopy is the MassARRAY® system developed by Sequenom.

A computer program may be utilized to carry out one or more steps of any of the methods disclosed herein. Another aspect of the present disclosure is a computer program product comprising a computer readable storage medium having a computer program stored thereon which, when loaded into a computer, performs or assists in the performance of any of the methods disclosed herein.

One aspect of the disclosure is a product of any of the methods disclosed herein, namely, an assay result, which may be evaluated at the site of the testing or it may be shipped to another site for evaluation and communication to an interested party at a remote location, if desired. As used herein, “remote location” refers to a location that is physically different than that at which the results are obtained. Accordingly, the results may be sent to a different room, a different building, a different part of city, a different city, and so forth. The data may be transmitted by any suitable means such as, e.g., facsimile, mail, overnight delivery, e-mail, ftp, voice mail, and the like.

“Communicating” information refers to the transmission of the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data.

### Modified Nucleotides

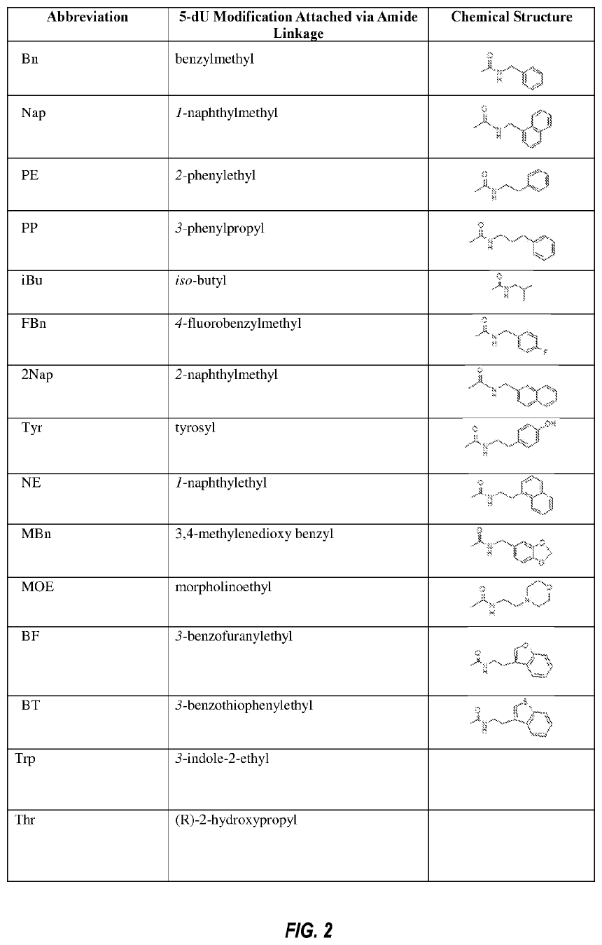

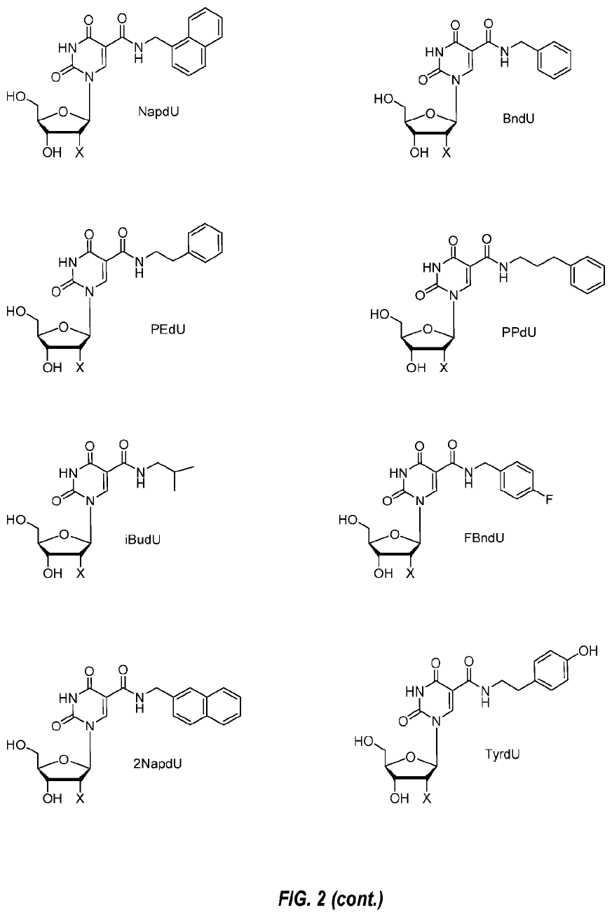

In certain embodiments, the disclosure provides oligonucleotides, such as aptamers, which comprise two different types of base-modified nucleotides. In some embodiments, the oligonucleotides comprise two different types of 5-position modified pyrimidines. In some embodiments, the oligonucleotide comprises at least one C5-modified cytidine and at least one C5-modified uridine. In some embodiments, the oligonucleotide comprises two different C5-modified cytidines. In some embodiments, the oligonucleotide comprises two different C5-modified uridines. Nonlimiting exemplary C5-modified uridines and cytidines are shown, for example, in FIG. 1. Certain nonlimiting exemplary C5-modified uridines are shown in FIG. 2, and certain non-limiting exemplary C5-modified cytidines are shown in FIG. 3.

### Preparation of Oligonucleotides

The automated synthesis of oligodeoxynucleosides is routine practice in many laboratories (see e.g., Matteucci, M. D. and Caruthers, M. H., (1990) J. Am. Chem. Soc., 103:3185-3191, the contents of which are hereby incorporated by reference in their entirety). Synthesis of oligoribonucleosides is also well known (see e.g. Scaringe, S. A., et al., (1990) Nucleic Acids Res. 18:5433-5441, the contents of which are hereby incorporated by reference in their entirety). As noted herein, the phosphoramidites are useful for incorporation of the modified nucleoside into an oligonucleotide by chemical synthesis, and the triphosphates are useful for incorporation of the modified nucleoside into an oligonucleotide by enzymatic synthesis. (See e.g., Vaught, J. D. et al. (2004) J. Am. Chem. Soc., 126:11231-11237; Vaught, J. V., et al. (2010) J. Am. Chem. Soc. 132, 4141-4151; Gait, M. J. “Oligonucleotide Synthesis a practical approach” (1984) IRL Press (Oxford, UK); Herdewijn, P. “Oligonucleotide Synthesis” (2005) (Humana Press, Totowa, N.J. (each of which is incorporated herein by reference in its entirety).

“Target” or “target molecule” or “target” refers herein to any compound upon which a nucleic acid can act in a desired or intended manner. A target molecule can be a protein, peptide, nucleic acid, carbohydrate, lipid, polysaccharide, glycoprotein, hormone, receptor, antigen, antibody, virus, pathogen, toxic substance, substrate, metabolite, transition state analog, cofactor, inhibitor, drug, dye, nutrient, growth factor, cell, tissue, any portion or fragment of any of the foregoing, etc., without limitation. Virtually any chemical or biological effector may be a suitable target. Molecules of any size can serve as targets. A target can also be modified in certain ways to enhance the likelihood or strength of an interaction between the target and the nucleic acid. A target can also include any minor variation of a particular compound or molecule, such as, in the case of a protein, for example, minor variations in amino acid sequence, disulfide bond formation, glycosylation, lipidation, acetylation, phosphorylation, or any other manipulation or modification, such as conjugation with a labeling component, which does not substantially alter the identity of the molecule. A “target molecule” or “target” is a set of copies of one type or species of molecule or multimolecular structure that is capable of binding to an aptamer. “Target molecules” or “targets” refer to more than one such set of molecules. Embodiments of the SELEX process in which the target is a peptide are described in U.S. Pat. No. 6,376,190, entitled “Modified SELEX Processes Without Purified Protein.” In some embodiments, a target is a protein.

As used herein, “competitor molecule” and “competitor” are used interchangeably to refer to any molecule that can form a non-specific complex with a non-target molecule. In this context, non-target molecules include free aptamers, where, for example, a competitor can be used to inhibit the aptamer from binding (rebinding), non-specifically, to another non-target molecule. A “competitor molecule” or “competitor” is a set of copies of one type or species of molecule. “Competitor molecules” or “competitors” refer to more than one such set of molecules. Competitor molecules include, but are not limited to oligonucleotides, polyanions (e.g., heparin, herring sperm DNA, salmon sperm DNA, tRNA, dextran sulfate, polydextran, abasic phosphodiester polymers, dNTPs, and pyrophosphate). In various embodiments, a combination of one or more competitor can be used.

As used herein, “non-specific complex” refers to a non-covalent association between two or more molecules other than an aptamer and its target molecule. A non-specific complex represents an interaction between classes of molecules. Non-specific complexes include complexes formed between an aptamer and a non-target molecule, a competitor and a non-target molecule, a competitor and a target molecule, and a target molecule and a non-target molecule.

In another embodiment, a polyanionic competitor (e.g., dextran sulfate or another polyanionic material) is used in the slow off-rate enrichment process to facilitate the identification of an aptamer that is refractory to the presence of the polyanion. In this context, “polyanionic refractory aptamer” is an aptamer that is capable of forming an aptamer/target complex that is less likely to dissociate in the solution that also contains the polyanionic refractory material than an aptamer/target complex that includes a nonpolyanionic refractory aptamer. In this manner, polyanionic refractory aptamers can be used in the performance of analytical methods to detect the presence or amount or concentration of a target in a sample, where the detection method includes the use of the polyanionic material (e.g. dextran sulfate) to which the aptamer is refractory.

Thus, in one embodiment, a method for producing a polyanionic refractory aptamer is provided. In this embodiment, after contacting a candidate mixture of nucleic acids with the target. The target and the nucleic acids in the candidate mixture are allowed to come to equilibrium. A polyanionic competitor is introduced and allowed to incubate in the solution for a period of time sufficient to insure that most of the fast off rate aptamers in the candidate mixture dissociate from the target molecule. Also, aptamers in the candidate mixture that may dissociate in the presence of the polyanionic competitor will be released from the target molecule. The mixture is partitioned to isolate the high affinity, slow off-rate aptamers that have remained in association with the target molecule and to remove any uncomplexed materials from the solution. The aptamer can then be released from the target molecule and isolated. The isolated aptamer can also be amplified and additional rounds of selection applied to increase the overall performance of the selected aptamers. This process may also be used with a minimal incubation time if the selection of slow off-rate aptamers is not needed for a specific application.

### Salts

It may be convenient or desirable to prepare, purify, and/or handle a corresponding salt of the compound, for example, a pharmaceutically-acceptable salt. Examples of pharmaceutically acceptable salts are discussed in Berge et al. (1977) “Pharmaceutically Acceptable Salts” J. Pharm. Sci. 66:1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R^{X+}, NH₂R^{X}₂⁺, NHR^{X}₃⁺, NR^{X} ₄⁺). Examples of some suitable substituted ammonium ions are those derived from: ethylamine, diethylamine, dicyclohexylamine, triethylamine, butylamine, ethylenediamine, ethanolamine, diethanolamine, piperizine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Other Embodiments

In some embodiments, a method is disclosed comprising a) contacting a first test sample with a first set of aptamers to form a first mixture, wherein the first test sample is a Z% dilution of the biological sample, wherein Z is from a 5% to 39% (or 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39) dilution of a biological sample, and there are at least A₃ different aptamers in the first set of aptamers; b) contacting a second test sample with a second set of aptamers to form a second mixture, wherein the second test sample is a Y% dilution of the biological sample, wherein Y is less than Z, and wherein there are at least A₂ different aptamers in the second set of aptamers; c) contacting a third test sample with a third set of aptamers to form a third mixture, wherein the third test sample is a X% dilution of the biological sample, wherein X is less than Y, and there are at least A₁ different aptamers in the third set of aptamers; d) incubating the first, second and third mixtures to allow for the formation of aptamer-protein complexes, and removing a majority of the aptamers that did not form aptamer-protein complexes; e) collecting the aptamers from the aptamer-protein complexes by dissociating the aptamer-protein complexes; f) detecting or quantifying the collected aptamers; wherein, a majority of the aptamers of the first set of aptamers, second set of aptamers and third set of aptamers each have affinity for a different target protein in the test sample, and are capable of forming a aptamer-protein complex with its target protein, and wherein A₃ is greater than A_{2,} and A₂ is greater than A_{2;} and wherein the sum of A₁, A₂ and A₃ is at least 4,000.

In one aspect, Z is from 10% to 30%, or from 15% to 25%, or about 20%.

In one aspect, Y is from 0.01% to 1% (or 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.6, 0.7, 0.8, 0.9 or 1) or from 0.1% to 0.8% or from 0.2% to 0.75 or about 0.5%.

In one aspect, X is from 0.001% to 0.009% (or 0.001, 0.002, 0.003, 0.004, 0.005, 0.006, 0.007, 0.008 or 0.009) or from 0.002% to 0.008% or from 0.003% to 0.007% or about 0.005%.

In one aspect, sum of A₁, A₂ and A₃ is at least 4,500 or 5,000.

In one aspect, A₃ is from 50% to 90% (or 50%, 55%, 60%, 65%, 70%, 75%, 80%, 85% or 90%) of the sum of A₁, A₂ and A₃; or from 60% to 85% of the sum of A₁, A₂ and A₃; or about 80% or 81% of the sum of A₁, A₂ and A₃.

In one aspect, A₂ is from 10% to 49% (or 10%, 15%, 20%, 25%, 30%, 35%, 40%, 45% or 49%) of the sum of A₁, A₂ and A₃; or from 12% to 35% of the sum of A₁, A₂ and A₃; or from 15% to 30% of the sum of A₁, A₂ and A₃; or about 15% or 16% of the sum of A₁, A₂ and A₃.

In one aspect, A₁ is from 1% to 9% (or 1%, 2%, 3%, 4%, 5%, 6%, 7%, 8% or 9%) of the sum of A₁, A₂ and A₃; or from 2% to 7% of the sum of A₁, A₂ and A₃; or from 3% to 6% of the sum of A₁, A₂ and A₃; or about 3% or 4% of the sum of A₁, A₂ and A₃.

In one aspect, A₃ is at least 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4200, 4270, 4500, 5000 (or is from 900 to 16,500 or from 2000 to 15,000 or from 3,000 to 12,000 or from 4,000 to 10,000).

In one aspect, A₂ is at least 200, 250, 300, 350, 400, 450, 500, 550, 600, 650, 700, 750, 800, 820, 900 (or is from 500 to 3500 or from 700 to 2500, or from 800 to 2000).

In one aspect, A₁ is at least 100, 110, 120, 130, 140, 150, 160, 170, 173 (or is from 100 to 700 or 100 to 650).

In one aspect, the first mixture, second mixture and third mixture are incubated separately from one another.

In one aspect, the methods herein further comprise combining the first mixture, second mixture and third mixture together after the mixtures are incubated to allow for aptamer-protein complex formation.

In one aspect, the methods herein further comprise sequentially combining the first mixture, second and third mixture together after the mixtures are incubated to allow for aptamer-protein complex formation.

In one aspect, the sequential combining is performed in an order selected from i) the first mixture, followed by the second mixture, followed by the third mixture; ii) the first mixture, followed by the third mixture, followed by the second mixture; iii) the second mixture, followed by the first mixture, followed by the third mixture; iv) the second mixture, followed by the third mixture, followed by the first mixture; v) the third mixture, followed by the second mixture, followed by the first mixture; and vi) the third mixture, followed by the first mixture, followed by the second mixture.

In one aspect, the test sample is selected from blood, plasma, serum sputum, breath, urine, semen, saliva, meningeal fluid, amniotic fluid, glandular fluid, lymph fluid, nipple aspirate, bronchial aspirate, synovial fluid, joint aspirate, cells, a cellular extract, and cerebrospinal fluid.

In one aspect, the detecting or quantifying is performed by PCR, mass spectrometry, nucleic acid sequencing, next-generation sequencing (NGS) or hybridization.

In one aspect, the at least A₃ different aptamers are differ from one another by at least one nucleotide differences and/or at least one nucleotide modification.

In one aspect, the at least A₂ different aptamers are differ from one another by at least one nucleotide differences and/or at least one nucleotide modification.

In one aspect, the at least A₁ different aptamers are differ from one another by at least one nucleotide differences and/or at least one nucleotide modification.

In one aspect, the at least A₃ different aptamers, the at least A₂ different aptamers and the at least A₁ different aptamers are differ from one another by at least one nucleotide differences and/or at least one nucleotide modification.

The methods of anyone of the proceeding paragraphs, wherein one or more aptamers of the first set, second set and third set of aptamers comprise at least one 5-position modified pyrimidine.

In one aspect, the at least one 5-positon modified pyrimidine comprises a linker at the 5-position of the pyrimidine and a moiety attached to the linker.

In one aspect, the linker is selected from amide linker, a carbonyl linker, a propynyl linker, an alkyne linker, an ester linker, a urea linker, a carbamate linker, a guanidine linker, an amidine linker, a sulfoxide linker, and a sulfone linker.

In one aspect, the moiety is a hydrophobic moiety.

In one aspect, the moiety is selected from the moieties of Groups I, II, III, IV, V, VII, VIII, IX, XI, XII, XIII, XV and XVI of FIG. 1.

In one aspect, the moiety is selected from a naphthyl moiety, a benzyl moiety, a fluorobenzyl moiety, a tyrosyl moiety, an indole moiety a morpholino moiety, an isobutyl moiety, a 3,4-methylenedioxy benzyl moiety, a benzothiophenyl moiety, and a benzofuranyl moiety.

In one aspect, the pyrimidine of the 5-position modified pyrimidine is a uridine, cytidine or thymidine.

In some embodiments, a method is disclosed comprising a) contacting a first test sample with at least one first aptamer to form a first mixture, wherein the first test sample is at least a X% dilution of a test sample; b) contacting a second test sample with at least one second aptamer to form a second mixture, wherein the second test sample is a Y% dilution of the test sample, wherein X is less than Y; c) contacting a third test sample with at least one third aptamer to form a third mixture, wherein the third test sample is a Z% dilution of the test sample, wherein Y is less than Z; d) incubating the first, second and third mixtures to allow for the formation of aptamer-protein complexes, and removing a majority of the aptamers that did not form aptamer-protein complexes; e) collecting the aptamers from the aptamer-protein complexes by dissociating the aptamer-protein complexes; f) detecting or quantifying the collected aptamers; wherein, the at least one first aptamer, the at least one second aptamer and the at least one third aptamer, each have affinity for a different protein, and are capable of forming an aptamer-protein complex when the protein is present in the respective test sample; wherein, the first, second and third test samples are a different dilution of the same test sample.

In one aspect, Z% is from 5% to 39% (or 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39%) or from 10% to 30% or from 15% to 25% or about 20%.

In one aspect, Y% is from 0.01% to 1% (or 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.6, 0.7, 0.8, 0.9 or 1%) or from 0.1% to 0.8% or from 0.2% to 0.7% or about 0.5%.

In one aspect, X% is from 0.001% to 0.009% (or 0.001, 0.002, 0.003, 0.004, 0.005, 0.006, 0.007, 0.008 or 0.009) or from 0.002% to 0.008% or from 0.003% to 0.007% or about 0.005%.

In one aspect, the first mixture comprises a plurality of aptamers.

In one aspect, the first mixture comprises at least 100, 110, 120, 130, 140, 150, 160, 170, 173 (or is from 100 to 700 or 100 to 650) different aptamers.

In one aspect, the second mixture comprises a plurality of aptamers.

In one aspect, the second mixture comprises at least at least 200, 250, 300, 350, 400, 450, 500, 550, 600, 650, 700, 750, 800, 820, 900 (or is from 500 to 3500 or from 700 to 2500, or from 800 to 2000) different aptamers.

In one aspect, the third mixture comprises a plurality of aptamers.

In one aspect, the third mixture comprises at least 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4200, 4270, 4500, 5000 (or is from 900 to 16,500 or from 2000 to 15,000 or from 3,000 to 12,000 or from 4,000 to 10,000) different aptamers.

In one aspect, the first mixture, second mixture and third mixture are incubated separately from one another.

In one aspect, the methods disclosed herein further comprise combining the first mixture, second mixture and third mixture together after the mixtures are incubated to allow for aptamer-protein complex formation.

In one aspect, the methods disclosed herein further comprise sequentially combining the first mixture, second and third mixture together after the mixtures are incubated to allow for aptamer-protein complex formation.

In one aspect, the sequential combining is performed in an order selected from i) the first mixture, followed by the second mixture, followed by the third mixture; ii) the first mixture, followed by the third mixture, followed by the second mixture; iii) the second mixture, followed by the first mixture, followed by the third mixture; iv) the second mixture, followed by the third mixture, followed by the first mixture; v) the third mixture, followed by the second mixture, followed by the first mixture; and vi) the third mixture, followed by the first mixture, followed by the second mixture.

In one aspect, the test sample is selected from blood, plasma, serum sputum, breath, urine, semen, saliva, meningeal fluid, amniotic fluid, glandular fluid, lymph fluid, nipple aspirate, bronchial aspirate, synovial fluid, joint aspirate, cells, a cellular extract, and cerebrospinal fluid.

In one aspect, the detecting or quantifying is performed by PCR, mass spectrometry, nucleic acid sequencing, next-generation sequencing (NGS) or hybridization.

The methods of anyone of the proceeding paragraphs, wherein the at least one first aptamer, the at least one second aptamer, the at least one third aptamer, and the plurality of aptamers comprise at least one 5-position modified pyrimidine.

In one aspect, wherein the at least one 5-positon modified pyrimidine comprises a linker at the 5-position of the pyrimidine and a moiety attached to the linker.

In one aspect, wherein the linker is selected from amide linker, a carbonyl linker, a propynyl linker, an alkyne linker, an ester linker, a urea linker, a carbamate linker, a guanidine linker, an amidine linker, a sulfoxide linker, and a sulfone linker.

In one aspect, wherein the moiety is a hydrophobic moiety.

In one aspect, wherein the moiety is selected from the moieties of Groups I, II, III, IV, V, VII, VIII, IX, XI, XII, XIII, XV and XVI of FIG. 1.

In one aspect, wherein the moiety is selected from a naphthyl moiety, a benzyl moiety, a fluorobenzyl moiety, a tyrosyl moiety, an indole moiety a morpholino moiety, an isobutyl moiety, a 3,4-methylenedioxy benzyl moiety, a benzothiophenyl moiety, and a benzofuranyl moiety.

In one aspect, the pyrimidine of the 5-position modified pyrimidine is a uridine, cytidine or thymidine.

The methods of anyone of the proceeding paragraphs, wherein the aptamers differ from one another by at least one nucleotide differences and/or at least one nucleotide modification.

In some embodiments, a system is disclosed comprising a) a first receptacle having a first mixture comprising a first test sample with a first set of aptamers, wherein the first test sample is an Z% dilution of a test sample, and there are at least A₃ different aptamers in the first set of aptamers; b) a second receptacle having a second mixture comprising a second test sample with a second set of aptamers, wherein the second test sample is a Y% dilution of the test sample, wherein Y is less than Z, and there are at least A₂ different aptamers in the second set of aptamers; c) a third receptacle having a third mixture comprising a third test sample with a third set of aptamers, wherein the third test sample is a X% dilution of the test sample, wherein X is less than Y, and there are at least A₁ different aptamers in the third set of aptamers; and wherein, a majority of the aptamers of the first set of aptamers, second set of aptamers and third set of aptamers have affinity for a protein in the test sample, and are capable of forming a aptamer-protein complex, and wherein A₃ is greater than A_{2,} and A₂ is greater than A₁; and wherein the sum of A₁, A₂ and A₃ is at least 4,000; and wherein, the system is used to detect proteins in the test sample, and the first, second and third test samples are a different dilution of the same test sample.

In one aspect, Z% is from 5% to 39% (or 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39%) or from 10% to 30% or from 15% to 25% or about 20%.

In one aspect, Y% is from 0.01% to 1% (or 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.6, 0.7, 0.8, 0.9 or 1%) or from 0.1% to 0.8% or from 0.2% to 0.7% or about 0.5%.

In one aspect, X% is from 0.001 to 0.009% (or 0.001, 0.002, 0.003, 0.004, 0.005, 0.006, 0.007, 0.008 or 0.009%) or from 0.002% to 0.008% or from 0.003% to 0.007% or about 0.005%.

In some embodiments, a system is disclosed comprising a) a first receptacle having a first mixture comprising a first test sample with at least one first aptamer, wherein the first test sample is an Z% dilution of a test sample; b) a second receptacle having a second mixture comprising a second test sample with at least one second aptamer, wherein the second test sample is a Y% dilution of the test sample, wherein Y is less than Z; c) a third receptacle having a third mixture comprising a third test sample with at least one third aptamer, wherein the third test sample is a X% dilution of the test sample, wherein X is less than Y; wherein, the at least one first aptamer, the at least one second aptamer and the at least one third aptamer, each have affinity for a different protein, and are capable of forming an aptamer-protein complex when the protein is present in the biological sample; and wherein, the system is used to detect proteins in the test sample, and the first, second and third test samples are a different dilution of the same test sample.

In one aspect, Z% is from 5% to 39% (or 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39%) or from 10% to 30% or from 15% to 25% or about 20%.

In one aspect, Y% is from 0.01% to 1% (or 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.6, 0.7, 0.8, 0.9 or 1%) or from 0.1% to 0.8% or from 0.2% to 0.7% or about 0.5%.

In one aspect, X% is from 0.001 to 0.009% (or 0.001, 0.002, 0.003, 0.004, 0.005, 0.006, 0.007, 0.008 or 0.009%) or from 0.002% to 0.008% or from 0.003% to 0.007% or about 0.005%.

In some embodiments, a formulation is disclosed comprising a first capture reagent-target molecule affinity complex, a second capture reagent-target molecule affinity complex and a third capture reagent-target molecule affinity complex, wherein the first capture reagent-target molecule affinity complex formed in about a 0.005% dilution of a test sample, the second capture reagent-target molecule affinity complex formed in about a 0.5% dilution of the test sample, and the third capture reagent-target molecule affinity complex formed in about a 20% dilution of the test sample.

In one aspect, independently, the first capture reagent of the first capture reagent-target molecule affinity complex, the second capture reagent of the second capture reagent-target molecule affinity complex, and the third capture reagent of the third capture reagent-target molecule affinity complex are selected from an aptamer or antibody.

In one aspect, the test sample is selected from plasma, serum, urine, whole blood, leukocytes, peripheral blood mononuclear cells, buffy coat, sputum, tears, mucus, nasal washes, nasal aspirate, semen, saliva, peritoneal washings, ascites, cystic fluid, meningeal fluid, amniotic fluid, glandular fluid, lymph fluid, nipple aspirate, bronchial aspirate, bronchial brushing, synovial fluid, joint aspirate, organ secretions, cells, a cellular extract, and cerebrospinal fluid.

In one aspect, target molecule of each of the first capture reagent-target molecule affinity complex, the second capture reagent-target molecule affinity complex and the third capture reagent-target molecule affinity complex is selected from a protein, a peptide, a carbohydrate, a polysaccharide, a glycoprotein, a hormone, a receptor, an antigen, an antibody, a virus, a bacteria, a metabolite, a cofactor, an inhibitor, a drug, a dye, a nutrient, a growth factor, a cell and a tissue.

In one aspect, the first capture reagent-target molecule affinity complex, the second capture reagent-target molecule affinity complex and the third capture reagent-target molecule affinity complex are non-covalent complexes.

In one aspect, each of the first capture reagent-target molecule affinity complex, the second capture reagent-target molecule affinity complex and the third capture reagent-target molecule affinity complex formed in their respective dilutions of the test sample prior to being combined in the formulation.

In one aspect, the aptamer comprises at least one 5-position modified pyrimidine.

In one aspect, the at least one 5-positon modified pyrimidine comprises a linker at the 5-position of the pyrimidine and a moiety attached to the linker.

In one aspect, the linker is selected from amide linker, a carbonyl linker, a propynyl linker, an alkyne linker, an ester linker, a urea linker, a carbamate linker, a guanidine linker, an amidine linker, a sulfoxide linker, and a sulfone linker.

In one aspect, the moiety is a hydrophobic moiety.

In one aspect, the moiety is selected from the moieties of Groups I, II, III, IV, V, VII, VIII, IX, XI, XII, XIII, XV and XVI of FIG. 1.

In one aspect, the moiety is selected from a naphthyl moiety, a benzyl moiety, a fluorobenzyl moiety, a tyrosyl moiety, an indole moiety a morpholino moiety, an isobutyl moiety, a 3,4-methylenedioxy benzyl moiety, a benzothiophenyl moiety, and a benzofuranyl moiety.

In one aspect, the pyrimidine of the 5-position modified pyrimidine is a uridine, cytidine or thymidine.

In some embodiments, a formulation is disclosed comprising a plurality of first capture reagent-target molecule affinity complexes, a plurality of second capture reagent-target molecule affinity complexes and a plurality of third capture reagent-target molecule affinity complexes, wherein the plurality of the first capture reagent-target molecule affinity complexes formed in about a 0.005% dilution of a test sample, the plurality of the second capture reagent-target molecule affinity complexes formed in about a 0.5% dilution of the test sample, and the plurality of the third capture reagent-target molecule affinity complexes formed in about a 20% dilution of the test sample.

In one aspect, independently, the plurality of first capture reagents of the plurality of the first capture reagent-target molecule affinity complexes, the plurality of second capture reagents of the plurality of the second capture reagent-target molecule affinity complexes, and the plurality of the third capture reagents of the plurality of the third capture reagent-target molecule affinity complexes are selected from an aptamer or antibody.

In one aspect, the test sample is selected from plasma, serum, urine, whole blood, leukocytes, peripheral blood mononuclear cells, buffy coat, sputum, tears, mucus, nasal washes, nasal aspirate, semen, saliva, peritoneal washings, ascites, cystic fluid, meningeal fluid, amniotic fluid, glandular fluid, lymph fluid, nipple aspirate, bronchial aspirate, bronchial brushing, synovial fluid, joint aspirate, organ secretions, cells, a cellular extract, and cerebrospinal fluid.

In one aspect, target molecule of each of the first capture reagent-target molecule affinity complex, the second capture reagent-target molecule affinity complex and the third capture reagent-target molecule affinity complex is selected from a protein, a peptide, a carbohydrate, a polysaccharide, a glycoprotein, a hormone, a receptor, an antigen, an antibody, a virus, a bacteria, a metabolite, a cofactor, an inhibitor, a drug, a dye, a nutrient, a growth factor, a cell and a tissue.

In one aspect, the plurality of first capture reagent-target molecule affinity complexes, the plurality of second capture reagent-target molecule affinity complexes and the plurality of third capture reagent-target molecule affinity complexes are non-covalent complexes.

In one aspect, each of the plurality of first capture reagent-target molecule affinity complexes, the plurality of second capture reagent-target molecule affinity complexes and the plurality of third capture reagent-target molecule affinity complexes formed in their respective dilutions of the test sample prior to being combined in the formulation.

In one aspect, the aptamer comprises at least one 5-position modified pyrimidine.

In one aspect, the at least one 5-positon modified pyrimidine comprises a linker at the 5-position of the pyrimidine and a moiety attached to the linker.

In one aspect, the linker is selected from amide linker, a carbonyl linker, a propynyl linker, an alkyne linker, an ester linker, a urea linker, a carbamate linker, a guanidine linker, an amidine linker, a sulfoxide linker, and a sulfone linker.

In one aspect, the moiety is a hydrophobic moiety.

In one aspect, the moiety is selected from the moieties of Groups I, II, III, IV, V, VII, VIII, IX, XI, XII, XIII, XV and XVI of FIG. 1.

In one aspect, the moiety is selected from a naphthyl moiety, a benzyl moiety, a fluorobenzyl moiety, a tyrosyl moiety, an indole moiety a morpholino moiety, an isobutyl moiety, a 3,4-methylenedioxy benzyl moiety, a benzothiophenyl moiety, and a benzofuranyl moiety.

In one aspect, the pyrimidine of the 5-position modified pyrimidine is a uridine, cytidine or thymidine.

In one aspect, the plurality of first capture reagents of the plurality of the first capture reagent-target molecule affinity complexes is about 100, 110, 120, 130, 140, 150, 160, 170 or 173; or is from 100 to 700; or from 100 to 650 capture reagents.

In one aspect, the plurality of second capture reagents of the plurality of the second capture reagent-target molecule affinity complexes is about 200, 250, 300, 350, 400, 450, 500, 550, 600, 650, 700, 750, 800, 820 or 900; or is from 500 to 3500; or is from about 700 to 2500; or is from 800 to 2000; or about 828 capture reagents.

In one aspect, the plurality of the third capture reagents of the plurality of the third capture reagent-target molecule affinity complexes is about 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4200, 4270, 4500 or 5000; or is from about 900 to 16,500; or from about 2000 to 15,000; or from about 3,000 to 12,000; or from about 4,000 to 10,000; or about 4271 capture reagents.

In some embodiments, a method is disclosed comprising a) sequentially combining a first dilution group with a second dilution group, wherein the first dilution group is an X% dilution of a test sample and comprises a first capture reagent bound to a first target protein forming a first capture reagent-target protein affinity complex, the second dilution group is a Y% dilution of the test sample and comprises a second capture reagent bound to a second target protein forming a second capture reagent-target protein affinity complex, and wherein the first and second target proteins are different proteins, and wherein X is less than Y; b) dissociating the capture reagents from their respective capture reagent-target protein affinity complexes; and c) detecting for the presence of or determining the level of the dissociated capture reagents.

In some aspect of the methods disclosed herein, the methods further comprise a sequential combining of a third dilution group with the first and second dilution groups, wherein the third dilution group is a Z% dilution of the test sample and comprises a third capture reagent bound to a third target protein forming a third capture reagent-target protein affinity complex, wherein the third target protein is different from the first and second target proteins, wherein Y is less than Z.

In one aspect, the first capture reagent and the second capture reagent are an aptamer or an antibody.

In one aspect, the first dilution and the second dilution groups are dilutions of the same test sample

In one aspect, the test sample is selected from plasma, serum, urine, whole blood, leukocytes, peripheral blood mononuclear cells, buffy coat, sputum, tears, mucus, nasal washes, nasal aspirate, semen, saliva, peritoneal washings, ascites, cystic fluid, meningeal fluid, amniotic fluid, glandular fluid, lymph fluid, nipple aspirate, bronchial aspirate, bronchial brushing, synovial fluid, joint aspirate, organ secretions, cells, a cellular extract, and cerebrospinal fluid.

In one aspect, the third dilution group is a different dilution of the same test sample, and/or wherein the third capture reagent is an aptamer or antibody.

In one aspect, the first and second capture reagent-target protein affinity complexes are non-covalent complexes.

In one aspect, the first dilution group is a dilution of the test sample of from 0.001% to 0.009% (or wherein X% is 0.001%, 0.002%, 0.003%, 0.004%, 0.005%, 0.006%, 0.007%, 0.008% or 0.009%) or X% is from 0.002% to 0.008% or X% is from 0.003% to 0.007% or X% is about 0.005%.

In one aspect, the second dilution group is a dilution of the test sample of from 0.01% to 1% (or wherein Y% is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or Y% is from 0.1% to 0.8% or Y% is from 0.2% to 0.75% or Y% is about 0.5%.

In one aspect, the third dilution group is a dilution of the test sample of from 5% to 39% (or Z% is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or Z% is from 15% to 30%, or Z% is from 15% to 25%, or Z% is about 20%.

In one aspect, the first dilution group further comprises a plurality of first capture reagents.

In one aspect, the second dilution group further comprises a plurality of second capture reagents.

In one aspect, the third dilution group further comprises a plurality of third capture reagents.

In one aspect, the first dilution group further comprises a plurality of first capture reagent-target protein affinity complexes.

In one aspect, the second dilution group further comprises a plurality of second capture reagent-target protein affinity complexes.

In one aspect, the third dilution group further comprises a plurality of third capture reagent-target protein affinity complexes.

In one aspect, the sequential combining of the first dilution group with the second dilution group further comprises a wash step after combining the first and second dilution groups.

In one aspect, the sequential combining of the third dilution group with the first and second dilution groups further comprises a wash step after combining the first, second and third dilution groups.

In one aspect, the plurality of first capture reagents is about 100, 110, 120, 130, 140, 150, 160, 170 or 173; or is from 100 to 700; or from 100 to 650 capture reagents.

In one aspect, the plurality of second capture reagents is about 200, 250, 300, 350, 400, 450, 500, 550, 600, 650, 700, 750, 800, 820 or 900; or is from 500 to 3500; or is from about 700 to 2500; or is from 800 to 2000; or about 828 capture reagents.

In one aspect, the plurality of third capture reagents is about 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4200, 4270, 4500 or 5000; or is from about 900 to 16,500; or from about 2000 to 15,000; or from about 3,000 to 12,000; or from about 4,000 to 10,000; or about 4271 capture reagents.

In one aspect, prior to the sequential combining of the first and second dilution groups, the first capture reagent-target protein affinity complex of the first dilution group and the second capture reagent-target protein affinity complex of the second dilution group are each immobilized on a first solid support in their respective dilution groups, and released from the first solid support to sequentially combine.

In one aspect, prior to the sequential combining of the third dilution group with the first and second dilution groups, the third capture reagent-target protein affinity complex of the third dilution group is immobilized on a first solid support in its respective dilution group, and released from the first solid support to sequentially combine.

In one aspect, the first capture reagent-target protein affinity complex was immobilized on its first solid support by association of the capture reagent with the solid support.

In one aspect, the second capture reagent-target protein affinity complex was immobilized on its first solid support by association of the capture reagent with the solid support.

In one aspect, the third capture reagent-target protein affinity complex was immobilized on its first solid support by association of the capture reagent with the solid support.

In one aspect, the detecting for the presence or the determining of the level of the dissociated first and second capture reagents is performed by PCR, mass spectrometry, nucleic acid sequencing, next-generation sequencing (NGS) or hybridization.

In one aspect, the aptamer comprises at least one 5-position modified pyrimidine.

In one aspect, the at least one 5-positon modified pyrimidine comprises a linker at the 5-position of the pyrimidine and a moiety attached to the linker.

In one aspect, the linker is selected from amide linker, a carbonyl linker, a propynyl linker, an alkyne linker, an ester linker, a urea linker, a carbamate linker, a guanidine linker, an amidine linker, a sulfoxide linker, and a sulfone linker.

In one aspect, the moiety is a hydrophobic moiety.

In one aspect, the moiety is selected from the moieties of Groups I, II, III, IV, V, VII, VIII, IX, XI, XII, XIII, XV and XVI of FIG. 1.

In one aspect, the moiety is selected from a naphthyl moiety, a benzyl moiety, a fluorobenzyl moiety, a tyrosyl moiety, an indole moiety a morpholino moiety, an isobutyl moiety, a 3,4-methylenedioxy benzyl moiety, a benzothiophenyl moiety, and a benzofuranyl moiety.

In one aspect, the pyrimidine of the 5-position modified pyrimidine is a uridine, cytidine or thymidine.

In one aspect, the aptamer is 35-100 nucleotides in length.

In one aspect, the aptamer comprises a consensus protein binding domain.

In one aspect, the aptamer comprises 5-positon modified pyrimidines numbering 3-20.

In one aspect, the order of the sequential combining of the dilution groups is selected from combining the first dilution group with the second dilution group followed by the third dilution group; combining the first dilution group with the third dilution group followed by the second dilution group; combining the second dilution group with the third dilution group followed by the first dilution group; combining the second dilution group with the first dilution group followed by the third dilution group; combining the third dilution group with the first dilution group followed by the second dilution group; and combining the third dilution group with the second dilution group followed by the first dilution group.

In one aspect, the order of the sequential combining of the dilution groups is selected from combining the first dilution group with the second dilution group and combining the second dilution group with the first dilution group.

In one aspect, the detecting for the presence of or determining the level of the dissociated capture reagents is a surrogate for the detection for the presence of or the determining the level of the target protein.

In some embodiments, a method is disclosed comprising a) releasing a first capture reagent-target molecule affinity complex from a first solid support and transferring the first capture reagent-target molecule affinity complex to a first mixture; b) releasing a second capture reagent-target molecule affinity complex from a second solid support and transferring the second capture reagent-target molecule affinity complex to the first mixture, thus combining the first and second capture reagent-target molecule affinity complexes in the first mixture; c) attaching a first tag to the target molecule of the first and second capture reagent-target molecule affinity complexes; d) contacting the tagged first and second capture reagent-target molecule affinity complexes to one or more third solid support(s) such that the tag immobilizes the first and second capture reagent-target molecule affinity complexes to the one or more third solid support(s); e) dissociating the capture reagents from the first and second capture reagent-target molecule affinity complexes; and f) detecting for the presence of or determining the level of the dissociated capture reagents; wherein, the first capture reagent-target molecule affinity complex and the second capture reagent-target molecule affinity complex were each formed in a different dilution of the same test sample.

In some embodiments, a method is disclosed comprising a) contacting a first capture reagent immobilized on a first solid support with a first dilution to form a first mixture, and contacting a second capture reagent immobilized on a second solid support with a second dilution to form a second mixture, and wherein each of the first and second capture reagents are capable of binding to a target molecule; b) incubating the first mixture and second mixture separately, wherein a first capture reagent-target molecule affinity complex is formed in the first mixture if the target molecule to which the first capture reagent has affinity for is present in the first mixture, and wherein a second capture reagent-target molecule affinity complex is formed in the second mixture if the target molecule to which the second capture reagent has affinity for is present in the second mixture; c) releasing the first capture reagent-target molecule affinity complex from the first solid support and transferring the first capture reagent-target molecule affinity complex to a third mixture; d) releasing the second capture reagent-target molecule affinity complex from the second solid support; e) after step c), transferring the second capture reagent-target molecule affinity complex to the third mixture, thus combining the first and second capture reagent-target molecule affinity complexes in the third mixture; f) attaching a first tag to the target molecule of the first and second capture reagent-target molecule affinity complexes; g) contacting the tagged first and second capture reagent-target molecule affinity complexes to a third solid support such that the tag immobilizes the first and second capture reagent-target molecule affinity complexes to the third solid support; h) dissociating the capture reagents from their respective capture reagent-target molecule affinity complexes and i) detecting for the presence of or determining the level of the dissociated capture reagents; wherein, the first dilution and the second dilution are different dilutions of a test sample.

In some embodiments, a method is disclosed comprising a) releasing a first capture reagent-target molecule affinity complex from a first solid support and transferring the first capture reagent-target molecule affinity complex to a first mixture; b) releasing a second capture reagent-target molecule affinity complex from a second solid support and transferring the second capture reagent-target molecule affinity complex to the first mixture, thus combining the first and second capture reagent-target molecule affinity complexes; c) releasing a third capture reagent-target molecule affinity complex from a third solid support and transferring the third capture reagent-target molecule affinity complex to the first mixture, thus combining the first, second and third capture reagent-target molecule affinity complexes; d) attaching a first tag to the target molecule of the first, second, and third capture reagent-target molecule affinity complexes; e) contacting the tagged first, second, and third capture reagent-target molecule affinity complexes to one or more fourth solid support(s) such that the tag immobilizes the first, second and third capture reagent-target molecule affinity complexes to the one or more fourth solid support(s); f) dissociating the capture reagents from the first, second and third capture reagent-target molecule affinity complexes; and g) detecting for the presence of or determining the level of the dissociated capture reagents; wherein, the first capture reagent-target molecule affinity complex, the second capture reagent-target molecule affinity complex and the third capture reagent-target molecule affinity complex were each formed in a different dilution of the same test sample.

In some embodiments, a method is disclosed comprising a) releasing a first capture reagent-target molecule affinity complex from a first solid support and transferring the first capture reagent-target molecule affinity complex to a first mixture; b) releasing a second capture reagent-target molecule affinity complex from a second solid support and transferring the second capture reagent-target molecule affinity complex to the first mixture, thus combining the first and second capture reagent-target molecule affinity complexes in the first mixture; c) dissociating the capture reagents from the first and second capture reagent-target molecule affinity complexes; and f) detecting for the presence of or determining the level of the dissociated capture reagents; wherein, the first capture reagent-target molecule affinity complex and the second capture reagent-target molecule affinity complex were each formed in a different dilution of the same test sample.

In some embodiments, a method is disclosed comprising a) releasing a first capture reagent-target molecule affinity complex from a first solid support and transferring the first capture reagent-target molecule affinity complex to a first mixture; b) releasing a second capture reagent-target molecule affinity complex from a second solid support and transferring the second capture reagent-target molecule affinity complex to the first mixture, thus combining the first and second capture reagent-target molecule affinity complexes in the first mixture; c) releasing a third capture reagent-target molecule affinity complex from a third solid support and transferring the third capture reagent-target molecule affinity complex to first mixture, thus combining the first, second and third capture reagent-target molecule affinity complexes in the first mixture; e) dissociating the capture reagents from the first, second and third capture reagent-target molecule affinity complexes; and f) detecting for the presence of or determining the level of the dissociated capture reagents; wherein, the first capture reagent-target molecule affinity complex, the second capture reagent-target molecule affinity complex and the third capture reagent-target molecule affinity complex were each formed in a different dilution of the same test sample.

In anyone of the methods, formulations and systems described herein, the methods, formulations and/or systems further comprises a competitor molecule.

In anyone of the methods, formulations and systems described herein, the methods, formulations and/or systems, the competitor molecule is at a concentration of from about 10 μM to about 120 μM (or 10, 15, 20, 25, 30, 35, 40, 45, 50, 55, 60, 65, 70, 75, 80, 85, 90, 95, 100, 105, 110, 115 or 120 μM); or from about 15 μM to about 80 μM; or about 20 μM; or about 30 μM or about 60 μM.

In anyone of the methods, formulations and systems described herein, the methods, formulations and/or systems, the competitor molecule is selected from oligonucleotides, polyanions, heparin, herring sperm DNA, salmon sperm DNA, tRNA, dextran sulfate, polydextran, abasic phosphodiester polymers, dNTPs, and pyrophosphate. In anyone of the methods, formulations and systems described herein, the methods, formulations and/or systems, the competitor molecule is an oligonucleotide comprising the nucleotide sequence of (A-C-BndU-BndU)₇AC.

In anyone of the methods, formulations and systems described herein, the methods, formulations and/or systems, the competitor molecule is at a concentration of about 30 μM for a test sample, wherein the test sample is plasma.

In anyone of the methods, formulations and systems described herein, the methods, formulations and/or systems, the competitor molecule is at a concentration of about 60 μM for a test sample, wherein the test sample is serum.

### EXAMPLES

The following examples are presented in order to more fully illustrate some embodiments of the invention. They should, in no way be construed, however, as limiting the broad scope of the invention. Those of ordinary skill in the art can readily adopt the underlying principles of this discovery to design various compounds without departing from the spirit of the current invention.

### Example 1

### Multiplexed Aptamer Analysis of Samples

This example describes the multiplex aptamer assay used to analyze samples and controls.

### Multiplex Aptamer Assay Method

All steps of the multiplex aptamer assay were performed at room temperature unless otherwise indicated.

### Preparation of Aptamer Master Mix Solutions.

5272 aptamers were grouped into three unique mixes, Dil1, Dil2 and Dil3 and corresponding to the plasma or serum sample dilutions of 20%, 0.5% and 0.005%, respectively. The assignment of an aptamer to a mix was empirically determined by assaying a dilution series of matching plasma and serum samples with each aptamer and identifying the sample dilution that gave the largest linear range of signal. The segregation of aptamers and mixing with different dilutions of plasma or serum sample (20%, 0.5% or 0.005%) allow the assay to span a 10⁷-fold range of protein concentrations. The stock solutions for aptamer master mix were prepared in HE-Tween buffer (10 mM Hepes, pH 7.5, 1 mM EDTA, 0.05% Tween 20) at 4 nM each aptamer and stored frozen at −20° C. 4271 aptamers were mixed in Dil1 mix, 828 aptamers in Dil2 and 173 aptamers in Dil3 mix. Before use, stock solutions were diluted in HE-Tween buffer to a working concentration of 0.55 nM each aptamer and aliquoted into individual use aliquots. Before using aptamer master mixes for Catch-0 plate preparation, working solutions were heat-cooled to refold aptamers by incubating at 95° C. for 10 minutes and then at 25° C. for at least 30 minutes before use.

Catch-0 plate preparation.

60 μL of Streptavidin Mag Sepharose 10% slurry (GE Healthcare, 28-9857) were combined with 100 μL of the heat-cooled aptamer master mix. The mixture was washed once with 175 μL of the Assay Buffer (40 mM HEPES, pH 7.5, 100 mM NaCl, 5 mM KCl, 5 mM MgCl₂, 1 mM EDTA, 0.05% Tween-20) and then dispensed to each well of a 96-well plate (Thermo Scientific, AB-0769). Plates were incubated for 30 minutes at 25° C. with shaking at 850 rpm on ThermoMixer C shaker (Eppendorf). After 30 min incubation, 6 μL of the MB Block buffer (50 mM D-Biotin in 50 mM Tris-HCl, pH 8, 0.01% Tween) was added to each well of the plate and plates were further incubated for 2 min with shaking. Plates were then washed with 175 μL of the Assay Buffer, wash cycle of 1 min shaking on the ThermoMixer C at 850 rpm followed by separation on the magnet for 30 seconds. After wash solution was removed, beads were resuspended in 175 μL of Assay buffer and stored at −20° C. until use.

### Catch-2 Bead Preparation.

Before the start of the robotic processing of the assay, 10 mg/mL bead slurry of MyOne Streptavidin C1 beads (Dynabeads, part number 35002D, Thermo Scientific) used for Catch-2 step of the multiplex aptamer assay was washed in bulk once the MB Prep buffer (10 mM Tris-HCl, pH8, 1 mM EDTA, 0.4% SDS) for 5 min followed by two washes with Assay buffer. After the last wash, beads were resuspended at 10 mg/mL concentration and 75 μL of bead slurry was dispensed into each well of the Catch-2 plate. At the beginning of the assay, Catch-2 plate was placed in the aluminum adapter and placed in the appropriate position on the Fluent deck.

### Sample Thawing and Dilutions.

65 μL aliquots of 100% plasma or serum samples, stored in Matrix tubes at −80° C., were thawed by incubating at room temperature for ten minutes. To facilitate thawing, the tubes were placed on top of the fan unit which circulated the air through the Matrix tube rack. After thawing the samples were centrifuged at 1000×g for 1 min and placed on the Fluent robot deck for sample dilution. A 20% sample solution was prepared by transferring 35 μL of thawed sample into 96-well plates containing 140 μL of the appropriate sample diluent. Sample diluent for plasma was 50 mM Hepes, pH 7.5, 100 mM NaCl, 8 mM MgCl₂, 5 mM KCl, 1.25 mM EGTA, 1.2 mM Benzamidine, 37.5 μM Z-Block and 1.2% Tween-20. Serum sample diluent contained 75 μM Z-block, the other components were the same concentration as in plasma sample diluent. Subsequent dilutions to make 0.5% and 0.005% diluted samples were made into Assay Buffer using serial dilutions on Fluent robot. To make 0.5% sample dilution, intermediate dilution of 20% sample to 4% was made by mixing 45 μL of 20% sample with 180 μL of Assay Buffer, then 0.5% sample was made by mixing 25 μL of 4% diluted sample with 175 μL of Assay Buffer. To make 0.005% sample, 0.05% intermediate dilution was made by mixing 20 μL of 0.5% sample with 180 μL of Assay Buffer, then 0.005% sample was made by mixing 20 μL of 0.05% sample with 180 μL of Assay Buffer.

### Sample Binding Step.

Catch-0 plates prepared by immobilizing the aptamer mixes on the Streptavidin Magnetic Sepharose beads as described above. Frozen plates were thawed for 30 min at 25° C. and were washed once with 175 82 L of Assay Buffer. 100 82 L of each sample dilution (20%, 0.5% and 0.005%) were added to the plates containing beads with three different aptamer master mixes (Dil1, Dil2 and Di13, respectively). Catch-0 plates were then sealed with aluminum foil seals (Microseal ‘F’ Foil, Bio-Rad) and placed in the 4-plate rotating shakers (PHMP-4, Grant Bio) set at 850 rpm, 28° C. Sample binding step was performed for 3.5 hours.

Multiplex aptamer assay processing on Fluent robot.

After sample binding step was completed, Catch-0 plates were placed into aluminum plate adapters and placed on the robot deck. Magnetic bead wash steps were performed using a temperature-controlled plate. For all robotic processing steps, the plates were set at 25° C. temperature except for Catch-2 washes as described below. Plates were washed 4 times with 175 μL of Assay Buffer, each wash cycle was programmed to shake the plates at 1000 rpm for at least 1 min followed by separation of the magnetic beads for at least 30 seconds before buffer aspiration. During the last wash cycle, the Tag reagent was prepared by diluting 100× Tag reagent (EZ-Link NHS-PEG4-Biotin, part number 21363, Thermo, 100 mM solution prepared in anhydrous DMSO) 1:100 in the Assay buffer and poured in the trough on the robot deck. 100 82 L of Tag reagent was added to each of the wells in the plates and incubated with shaking at 1200 rpm for 5 min to biotinylate proteins captured on the bead surface. Biotinylation reactions were quenched by addition of 175 82 L of Quench buffer (20 mM glycine in Assay buffer) to each well. Plates were incubated static for 3 min then washed 4 times with 175 82 L of Assay buffer, washes were performed under the same conditions as described above.

### Photo-Cleavage and Kinetic Challenge.

After the last wash of the plates, 90 82 L of Photocleavage buffer (2 μM of a oligonucleotide competitor in Assay buffer; the competitor has the nucleotide sequence of 5′-(AC-Bn-Bn)₇-AC-3′, where Bn indicates a 5-position benzyl-substituted deoxyuridine residue) was added to each well of the plates. The plates were moved to a photocleavage substation on the Fluent deck. The substation consists of the BlackRay light source (UVP XX-Series Bench Lamps, 365 nm) and three Bioshake 3000-T shakers (Q Instruments). Plates were irradiated for 20 min minutes with shaking at 1000 rpm.

### Catch-2 Bead Capture.

At the end of the photocleavage process, the buffer was removed from Catch-2 plate via magnetic separation, plate was washed once with 100 μL of Assay buffer. Photo-cleaved eluate containing aptamer-protein complexes was removed from each Catch-0 plate starting with the dilution 3 plate. All 90 μL of the solution was first transferred to the Catch-1 Eluate plate positioned on the shaker with raised magnets to trap any Streptavidin Magnetic Sepharose beads which might have been aspirated. After that, solution was transferred to the Catch-2 plate and the plate was incubated for 3 min with shaking at 1400 rpm at 25° C. After the incubation for 3 min, the magnetic beads were separated for 90 seconds, solution removed from the plate and photocleaved Dil2 plate solution was added to plate. Following identical process, the solution from Dil1 plate was added and incubated for 3 min. At the end of the 3 min incubation, 6 μL of the MB Block buffer was added to the magnetic bead suspension and beads were incubated for 2 min with shaking at 1200 rpm at 25° C. After this incubation, the plate was transferred to a different shaker which was preset to 38° C. temperature. Magnetic beads were separated for 2 minutes before removing the solution. Then, the Catch-2 plate was washed 4 times with 175 μL of MB Wash buffer (20% glycerol in Assay Buffer), each wash cycle was programmed to shake the beads at 1200 rpm for 1 min and allow the beads to partition on the magnet for 3.5 minutes. During the last bead separation step, the shaker temperature was set to 25° C. Then beads were washed once with 175 μL of Assay buffer. For this wash step, beads were shaken at 1200 rpm for 1 min and then allowed to separate on the magnet for 2 minutes. Following the wash step, aptamers were eluted from the purified aptamer-protein complexes using Elution buffer (1.8 M NaClO₄, 40 mM PIPES, pH 6.8, 1 mM EDTA, 0.05% Triton X-100). Elution was done using 75 μL of Elution buffer for 10 min at 25° C. shaking beads at 1250 rpm. 70 μL of the eluate was transferred to the Archive plate and separated on the magnet to partition any magnetic beads which might have been aspirated. 10 μL of the eluted material was transferred to the black half-area plate, diluted 1:5 in the Assay buffer and used to measure the Cyanine 3 fluorescence signals which are monitored as internal assay QC. 20 μL of the eluted material was transferred to the plate containing 5 μL of the Hybridization Blocking solution (Oligo aCGH/ChIP-on-chip Hybridization Kit, Large Volume, Agilent Technologies 5188-5380, containing a spike of Cyanine 3-labeled DNA sequence complementary to the corner marker probes on Agilent arrays). This plate was removed from the robot deck and further processed for hybridization (see below). Archive plate with the remaining eluted solution was heat-sealed using aluminum foil and stored at −20° C.

### Hybridization.

25 82 L of 2× Agilent Hybridization buffer (Oligo aCGH/ChIP-on-chip Hybridization Kit, Agilent Technologies, part number 5188-5380) was manually pipetted to the each well of the plate containing the eluted samples and blocking buffer. 40 82 L of this solution was manually pipetted into each “well” of the hybridization gasket slide (Hybridization Gasket Slide-8 microarrays per slide format, Agilent Technologies). Custom SurePrint G3 8x60k Agilent microarray slides containing 10 probes per array complementary to each aptamer were placed onto the gasket slides according to the manufacturer's protocol. Each assembly (Hybridization Chamber Kit-SureHyb enabled, Agilent Technologies) was tightly clamped and loaded into a hybridization oven for 19 hours at 55° C. rotating at 20 rpm.

### Post-Hybridization Washing.

Slide washing was performed using Little Dipper Processor (model 650C, Scigene). Approximately 700 mL of Wash Buffer 1 (Oligo aCGH/ChIP-on-chip Wash Buffer 1, Agilent Technologies) was poured into large glass staining dish and used to separate microarray slides from the gasket slides. Once disassembled, the slides were quickly transferred into a slide rack in a bath containing Wash Buffer 1 on the Little Dipper. The slides were washed for five minutes in Wash Buffer 1 with mixing via magnetic stir bar. The slide rack was then transferred to the bath with 37° C. Wash Buffer 2 (Oligo aCGH/ChIP-onchip Wash Buffer 2, Agilent Technologies) and allowed to incubate for five minutes with stirring. The slide rack was slowly removed from the second bath and then transferred to a bath containing acetonitrile and incubated for five minutes with stirring.

### Microarray Imaging.

The microarray slides were imaged with a microarray scanner (Agilent G4900DA Microarray Scanner System, Agilent Technologies) in the Cyanine 3-channel at 3μm resolution at 100% PMT setting and the 20-bit option enabled. The resulting tiff images were processed using Agilent Feature Extraction software (version 10.7.3.1 or higher) with the GE1_1200_Jun14 protocol.

### Example 2

### Non-Specific Target Molecule Capture in a Multiplex Assay

This example provides a description of non-specific target molecule capture and carry-over in a multi-catch multiplex assay.

Generally, the sensitivity and specificity of many assay formats are impacted by the ability of the detection method to resolve true signal from signal that arises due to nonspecific associations during the assay, which results in an unwanted detectable signal (false positive or assay “noise”). This is particularly true for multiplexed assays. It has been observed that one of the main sources of non-specific binding is a function of unanticipated capture-reagent-target molecule interactions. This example describes how non-specific capture-reagent-target molecule interactions may create unwanted signal or “noise” in an assay.

For this example, a aptamer based multiplex assay with a two-catch system and multiple dilutions of the test sample were used to model non-specific target molecule (e.g., protein) capture and carry-over due to unanticipated aptamer-target molecule interactions, which results in assay signals that fall outside the dynamic range of the assay, and decrease the sensitivity and specificity of the assay.

Briefly, the aptamer based assay was performed by incubating an aptamer reagent, which was immobilized to a first solid support (e.g., streptavidin-bead using a biotin on the reagent), with a biological sample (e.g., serum or plasma) and allowing the proteins in the biological sample to bind to their cognate aptamer (termed “catch-1”). A tag was then attached to the protein, and the aptamer-protein target complexes were then released from the first solid support, and exposed to a second solid support, whereby the aptamer-target protein complex was immobilized via the tag on the protein (termed “catch-2”). The complexes were then washed to remove any unbound aptamers and proteins from catch-2. After washing, the aptamer was released from the aptamer-target protein complex on the second solid support and captured for detection purposes (e.g., hybridization array). The quantification of the aptamer was used as a surrogate for the amount of protein in the biological sample. The aptamer based assay may be used with a single aptamer reagent or a plurality of aptamer reagents (or multiplex format).

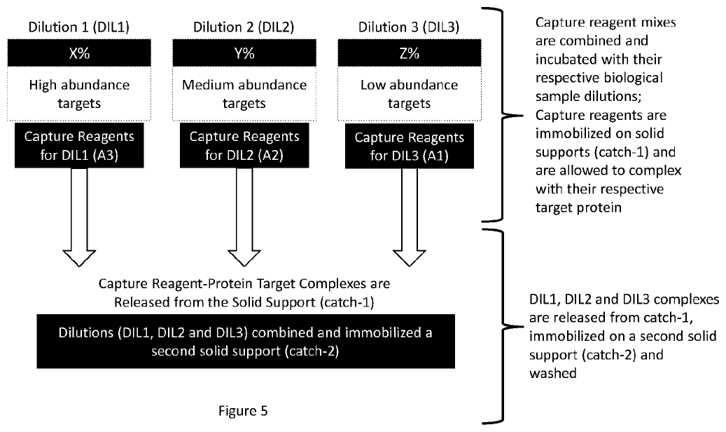

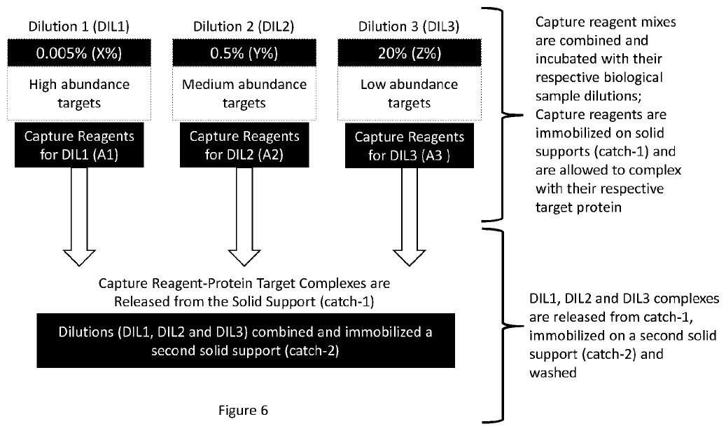

For this example, three different dilution groups of a plasma sample were made (serum was also subjected to the same “protein carry over study and the results parallel those of serum; data not shown). FIG. 6 provides an overview of the three different dilution groups of plasma that were made: a 0.005% dilution (DIL1), a 0.5% dilution (DIL2) and a 20% dilution (DIL3), where the relative high, medium and low abundance proteins were measured, respectively. Further, the aptamer sets for each of DIL1, DIL2 and DIL3 were A1, A₂ and A_{3,} respectively. The A₃ group of aptamers had 4,271 different aptamers (or ˜81% of the total number of aptamers), the A₂ group had 828 different aptamers (or ˜16% of the total number of aptamers) and the A1 group has 173 different aptamers (˜3% of the total number of aptamers) for a total of 5,272 different aptamers.

Five different conditions were tested to determine if there is a protein carryover effect in the multiplex assay. These conditions are shown in Table 2 below.

TABLE 2

| | DIL1 (0.005%) | DIL2 (0.5%) | DIL3 (20%) |
|---|---|---|---|
| Condition | or Blank1 | or Blank2 | or Blank3 |
| 1 | plasma | plasma | plasma |
| 2 | plasma | blank | blank |
| 3 | blank | plasma | blank |
| 4 | blank | blank | plasma |
| 5 | blank | blank | blank |

Each condition was subjected to the aptamer based multiplex assay with a two-catch system as described above. The conditions differ in whether or not a biological sample (e.g., plasma) was present or a blank, which was assay buffer with no biological sample and thus no protein. Each dilution group, irrespective of whether a diluted biological sample was present or a blank, was incubated with its respective group of aptamers (A1 with the DIL1 or Blank1; A2 with DIL2 or Blank2 and A3 with DIL3 or Blank3). In each case, the aptamers from each aptamer group were pre-immobilized on a first solid support prior to being incubated with their respective dilution or blank (catch-1). After incubation, a tag was then attached to the protein (if present), and the aptamer-protein target complexes (if present) were then released from the first solid support in the three separate dilutions and/or blanks and combined into a single mixture at the same time, and then exposed to a second solid support, whereby the aptamer-target protein complex (if present) was immobilized via the tag on the protein (termed “catch-2”). The complexes were then washed to remove any unbound aptamers and proteins from catch-2. After washing, the aptamer was released from the aptamer-target protein complex on the second solid support and captured for detection purposes via hybridization array. The quantification of the aptamer via relative fluorescent units (RFU's) was used as a surrogate for the amount of protein in the biological sample.

Condition 1 was plasma diluted into the three dilution groups (DIL1 at 0.005% dilution; DIL2 at 0.5% dilution and DIL3 at 20% dilution), which were incubated with their respective aptamers groups (A1, A2 and A3). Condition 2 had the DIL1 plasma dilution (0.005%) and Blank1 and Blank2 instead of DIL2 and DIL3, respectively, which were incubated with their respective aptamers groups (A1, A2 and A3). Condition 3 had the DIL2 plasma dilution (0.5%) and Blank 1 and Blank3 instead of DIL1 and DIL3, respectively, which were incubated with their respective aptamers groups (A1, A2 and A3). Condition 4 had the DIL3 plasma dilution (20%), and Blank 1 and Blank2 instead of DIL1 and DIL2, respectively, which were incubated with their respective aptamers groups (A1, A2 and A3). Lastly, Condition 5 had no plasma dilutions and had all blanks (Blank1, Blank2 and Blank3), which were incubated with their respective aptamers groups (A1, A2 and A3). Each condition was subjected to the catch-1 and catch-2 assay described in Example 1, whereby the dilution and/or blanks were combined all together after being released from catch-1 to move to the catch-2 part of the assay.

To quantify any protein carryover, the cumulative distribution function (CDF) of the ratio of the aptamer signal for Condition 1 (i.e., all three dilution groups DIL1, DIL2 and DIL3) to the aptamer signal for each of Conditions 2, 3 and 4 (where only one of the dilution groups was present along with blanks) was plotted (see FIG. 10). The ratio of aptamer signals are represented by relative fluorescent units (RFU's) derived from a hybridization array. FIG. 10 shows that for Condition 4, where only the 20% dilution (DIL3) of the plasma sample is present, that the ratio of the RFU values for the aptamers in Condition 1 to the same aptamers in Condition 4 is about 1. In contrast, for Condition 3, where only the 0.5% dilution (DIL2) of the plasma sample is present, the ratio of the RFU values for the aptamers of Condition 1 relative to the same aptamers in Condition 3 is from about 1 to 6, with about 45% or more of the aptamers of Condition 1 signaling at about 2 to 6 fold higher than the same aptamers for Condition 3. In looking at the signaling of a single aptamer under Condition 3 (e.g., the aptamer that binds protein ASM3A is part of the A2 group of aptamer, which is incubated with the DIL2 dilution) relative to Condition 1, the ASM3A aptamer is 5-folder higher in Condition 1 compared to Condition 3. For Condition 2, where only the 0.005% dilution (DIL1) of the plasma sample is present, the ratio of the RFU value for the aptamers of Condition 1 relative to the same aptamers in Condition 2 is also from about 1 to 6 fold, with about 20% or more of the aptamers of Condition 1 signaling at about 2 to 6 fold higher than the same aptamers for Condition 2. In comparing the aptamer that binds to the ApoE protein, which is part of the A1 aptamer group and incubated with the DIL1 dilution, this aptamer had an 200-fold greater RFU value in Condition 1 compared to Condition 2, an 80-fold greater RFU value in Condition 1 compared to Condition 4, and a 600-fold greater RFU value in Condition 1 compared to Condition 3.

These data show that the signal being detected in the assay, when all three dilutions samples are combined at the same time at the catch-2 phase of the assay, for the 0.5% plasma dilution sample (DIL2) and the 0.005% plasma dilution sample (DIL1) resulted from protein carry-over from the 20% plasma dilution sample (DIL3). This protein carry-over is likely due to proteins in the 20% plasma dilution sample (DIL3) being non-specifically bound to an aptamer in the A3 aptamer group, during the catch-1 phase of the assay, being released into solution by, for example, photocleavage from the first solid support (catch-1), and transferred to the catch-2 phase of the assay where all three dilution groups and aptamer groups are combined at the same time. At this phase of the assay, when a competitor is added to prevent non-specific aptamer-protein interactions, the proteins carried over non-specifically from the 20% plasma dilution are permitted to interact with the unbound aptamers from the A2 aptamer and A1 aptamer groups, and subsequently encounter their cognate aptamer to form stable complexes. These protein carry-over:aptamer complexes are then disrupted and the aptamer is detected on the hybridization array as a positive signal, which is technically a false positive signal or “noise”. These same data were observed with serum as the biological sample (data not shown).

These data indicate that a protein carry-over mitigation strategy is required to ensure that a multiplex assay remains within the dynamic range of the assay, and that the sensitivity and specificity of the assay is maximized.

### Example 3

### Mitigation Strategies to Reduce Non-Specific Target Molecule Capture in a Multiplex Assay

This example provides a description of an exemplary mitigation strategy to reduce non-specific target molecule capture and carry-over in a multi-catch multiplex assay.

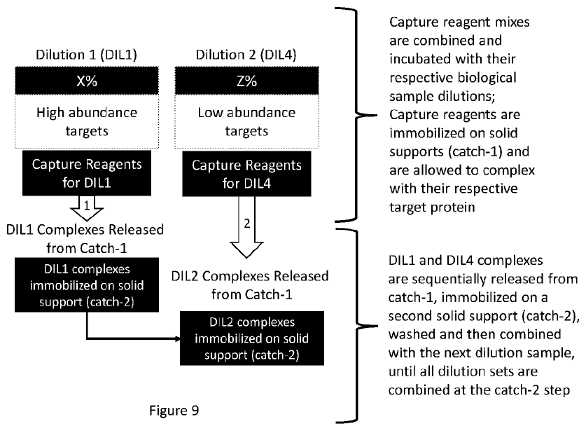

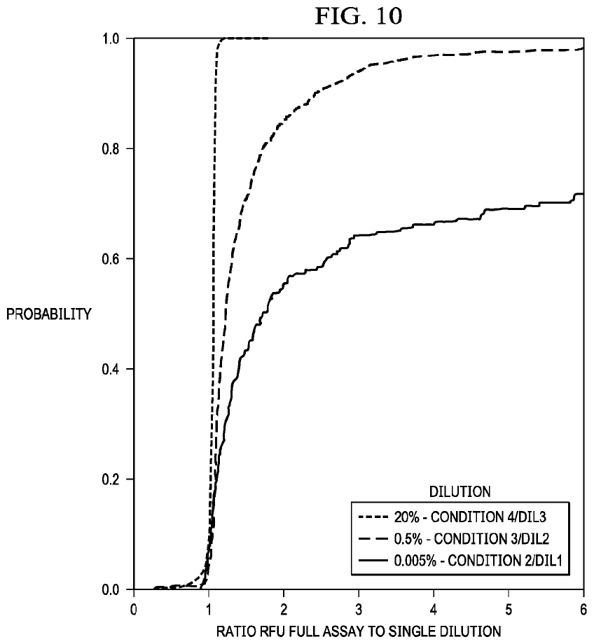

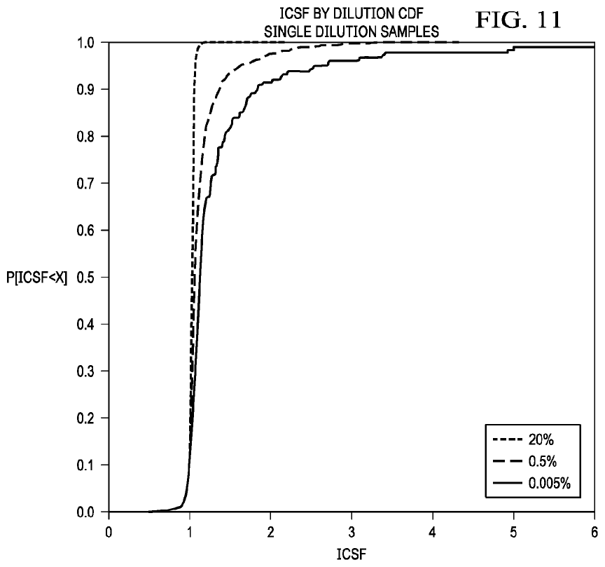

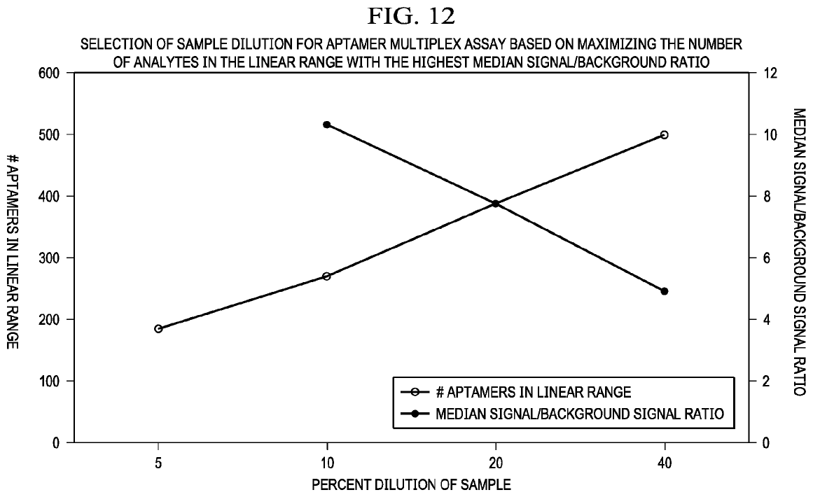

Example 1 provided a description of how positive signals in a multi-catch multiplex assay may be derived from non-specific target molecule capture and carry-over in the assay and its origin. In order to mitigate the unwanted protein carry-over in this multi-catch multiplex assay, a sequential release and catch of the dilution samples of the biological sample, along with the respective aptamer group, was performed in the course of transfer from the catch-1 phase of the assay to the catch-2 phase of the assay. A general overview of a two dilution and three dilution sequential catch format is shown in FIGS. 9 and 7, respectively.

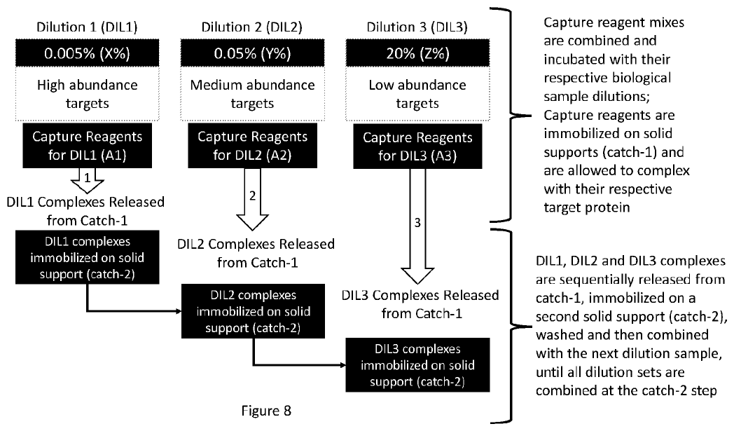

For this example, the same three different dilution group of plasma were made (DIL3, DIL2 and DIL1) along with the same aptamer groups (A1, A2 and A3) as was described in Example 1 (see FIG. 8). Further, the same conditions as described in Table 2 in Example 1 were used. Per Example 1, the same approach described for the catch-1 phase of the assay was followed; however, for this example, the different dilution groups or blanks were released individually and transferred to the catch-2 phase of the assay sequentially instead of at the same time per Example 1 (see FIG. 8). More specifically for Condition 1, the DIL1 group that was incubated with aptamer group A1 (DIL1-A1 group) was released from catch-1, and immobilized onto a second solid support (catch-2), and washed. Next, the DIL2 group that was incubated with aptamer group A2, was released from catch-1, combined with the DIL1-A1 group that was already immobilized on catch-2, and then immobilized onto a second solid support (catch-2). And, the DIL3 group that was incubated with aptamer group A3 was released from catch-1, and immobilized into a second solid support (catch-1), and washed. The reaming conditions (Conditions 2, 3, 4 and 5) that included a blank (Blank 1, 2 and/or 3) instead of a diluted biological sample, as outlined in Table 2, were subject to the same sequential catch approach.

To quantify any protein carryover, the cumulative distribution function (CDF) of the ratio of the aptamer signal for Condition 1 (i.e., all three dilution groups DIL1, DIL2 and DIL3) to the aptamer signal for each of Conditions 2, 3 and 4 (where only one of the dilution groups was present along with blanks) was plotted (see FIG. 11). The ratio of aptamer signals are represented by relative fluorescent units (RFU's) derived from a hybridization array. Similar to the non-sequential version of the multiplex assay, FIG. 11 shows that for Condition 4, where only the 20% dilution (DIL3) of the plasma sample is present, the ratio of the RFU values for the aptamers in Condition 1 to the same aptamers in Condition 4 is about 1. For Condition 3, where only the 0.5% dilution (DIL2) of the plasma sample is present, the ratio of the RFU values for the aptamers of Condition 1 relative to the same aptamers in Condition 3 is from about 1 to 6; however, only less than about 5% of the aptamers of Condition 1 signal at about 2 to 6 fold higher than the same aptamers for Condition 3 (versus 45% in the non-sequential catch-2 version of the assay). Further, for Condition 2, where only the 0.005% dilution (DIL1) of the plasma sample is present, the ratio of the RFU value for the aptamers of Condition 1 relative to the same aptamers in Condition 2 is also from about 1 to 6 fold; however, only less than about 10% of the aptamers of Condition 1 signaling at about 2 to 6 fold higher than the same aptamers for Condition 2 (versus 20% for the non-sequential catch-2 version of the assay). These same data were observed with serum as the biological sample (data not shown).

These data indicate that protein carry-over may be mitigated in a two-catch multiplex assay having two or more sample dilution sets by sequentially transferring the two or more diluted biological sample sets with its respective incubated capture reagents from the first catch phase of the assay to the second catch phase of the assay. This sequential transfer approach ensures that a multiplex assay remains within the dynamic range of the assay, that the sensitivity and specificity of the assay is maximized, and reduces potential false positive signals or “noise” in the assay.

### Example 4

### Dilution Selection for a Biological Sample to Maximize the Number of Analytes in the Linear Range Having the Highest Median Signal to Background Ratio in a Multiplex Assay

This example provides a description for selecting the dilution level of a biological sample that maximizes the number of analytes in the linear range while still maintaining the greatest median signal to background signal ratio in a multiplex assay.

In a multiplex assay format where multiple target proteins are being measured by multiple capture reagents, the natural variation in the abundance of the different target proteins can limit the ability of certain capture reagents to measure certain target proteins (e.g., high abundance target proteins may saturate the assay and prevent or reduce the ability of the assay to measure low abundance target proteins). To address this variation in the biological sample, the aptamer reagents are separated into at least two different groups, preferably three different groups, based on the abundance of their respective protein target in the biological sample. The biological sample is diluted into at least two, preferably three, different dilution groups to create separate test samples based on relative concentrations of the protein targets to be detected by their capture reagents. Thus, the biological sample is diluted into high, medium and low abundant target protein dilution groups, where the least abundant protein targets are measured in the least diluted group, and the most abundant protein targets are measured in the greatest diluted group. Historically for the aptamer based multi-catch multiplex assay, the three dilution groups for a biological sample were a 40% dilution, 1% dilution and a 0.005% dilution.

For this Example, the 40% dilution group was revisited to determine if a different dilution would provide greater benefit to the multi-catch multiplex assay (e.g., maximize the number of analytes in the linear range of the assay and/or improve the median signal to background signal ratio). This dilution group exhibits some non-specific binding, signal non-linearity and higher signals from negative controls compared to buffer alone.

Briefly, several dilution groups were made from plasma (a 40%, 20%, 10% and 5% dilution group) from three different subjects. A pool of 903 aptamers were incubated with the different dilution groups from all three subjects and used in the two-catch multiplex assay described herein.

The number of analytes in the linear range for each of the dilutions (40%, 20%, 10% and 5%) as measured by aptamers in the hybridization array was determined. For the 40% dilution, 246 analytes were in the linear range, for the 20% dilution, 388 analytes were in the linear range, for the 10% dilution, 517 analytes were in the linear range, and for the 5% dilution, 585 analytes were in the linear range. The remaining 259 of the 903 did not have a linear range. Thus, these data indicate that as the dilution of the sample increases the number of analytes in the linear range increase (i.e., a more dilute sample provides for a greater number of analytes in the linear range).

Each dilution (40%, 20%, 10% and 5%) exhibited a different median signal to background signal ratio (or Median S/B). For the 40% dilution, the Median S/B was 10, for the 20% dilution, the Median S/B was 7.8, for the 10% dilution, the Median S/B was 5.4 and for the 5% dilution, the Median S/B was 3.7. Thus, these data indicate that the Median S/B decreases as the sample is further diluted.

The data above indicates that there is a tension between the number of analytes in the linear range and the Median S/B related to the degree of sample dilution. In balancing the improvements observed to the number of analytes in the linear range with greater dilutions along with the greater Median S/B with lesser dilution, a “middle ground” was selected for the “optimal” dilution for the biological sample for the two-catch multiplex aptamer assay. FIG. 12 is a graphical representation of the number of analytes in the linear range along with the Median S/B for each of the dilutions of 40%, 20%, 10% and 5%. Per FIG. 12, at the 20% dilution of the biological sample, the maximum number of analytes in the linear range having the greatest Median S/B is observed (where the two lines intersect). Thus, of the three dilutions used in the multi-catch multiplex aptamer assay, the aptamers that target “low abundance” proteins are better suited to be incubated with a 20% dilution of the biological sample rather than a 40% dilution.

In summary, the multiplex assay described in the Examples section herein, uses the 20%, 0.5% and 0.005% sample dilution formats. Further, higher competitor molecule concentration in serum resulted in better correlations between the measurements in serum and plasma from the same individual (data not shown). In addition, a higher competitor molecule concentration (30 μM or 60 μM compared to 20 μM) with lower sample concentration (e.g., 40% to 20%) resulted in increased spike and recovery, an increase in the number of analytes in the linear range and less non-specific binding. The concentration of the competitor molecule (Z-block; oligonucleotide with the sequence ((A-C-BndU-BndU)₇AC) in the sample diluents was 60 μM for serum and 30 μM for plasma samples. Previous assay formats used 20 μM Z-block for serum and plasma. The higher competitor molecule concentration in serum resulted in better correlations between the measurements in serum and plasma from the same individual (data not shown). The decreased non-specific binding should result in a lower amount of proteins available for complex formation after photocleavage.

1. A method comprising:
a) contacting a first dilution sample with a first aptamer, wherein a first aptamer affinity complex is formed by the interaction of the first aptamer with its target molecule if the target molecule is present in the first dilution sample;
b) contacting a second dilution sample with a second aptamer, wherein a second aptamer affinity complex is formed by the interaction of the second aptamer with its target molecule if the target molecule is present in the second dilution sample;
c) incubating the first and second dilution samples separately to allow aptamer affinity complex formation;
d) transferring the first dilution sample with the first aptamer affinity complex to a first mixture, wherein the first aptamer affinity complex is captured on a solid support in the first mixture;
e) after step d), transferring the second dilution sample to the first mixture to form a second mixture, wherein the second aptamer affinity complex of the second dilution is captured on a solid support in the second mixture;
f) detecting for the presence of or determining the level of the first aptamer and second aptamer of the first and second aptamer affinity complexes, or the presence or amount of one or more first and second aptamer affinity complexes;
wherein, the first dilution and the second dilution are different dilutions of the same test sample, further comprising contacting a third dilution sample with a third aptamer, wherein a third aptamer affinity complex is formed by the interaction of the third aptamer with its target molecule if the target molecule is present in the third dilution sample.

2. The method of claim 1, wherein the test sample is selected from plasma, serum, urine, whole blood, leukocytes, peripheral blood mononuclear cells, buffy coat, sputum, tears, mucus, nasal washes, nasal aspirate, semen, saliva, peritoneal washings, ascites, cystic fluid, meningeal fluid, amniotic fluid, glandular fluid, lymph fluid, nipple aspirate, bronchial aspirate, bronchial brushing, synovial fluid, joint aspirate, organ secretions, cells, a cellular extract, and cerebrospinal fluid.

3. The method of claim 1, wherein the first and second aptamer-target molecule affinity complexes are non-covalent complexes.

4. The method of claim 1, wherein the target molecule is selected from a protein, a peptide, a carbohydrate, a polysaccharide, a glycoprotein, a hormone, a receptor, an antigen, an antibody, a virus, a bacteria, a metabolite, a cofactor, an inhibitor, a drug, a dye, a nutrient, a growth factor, a cell and a tissue.

5. The method of claim 1, wherein the first dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008% or is from 0.003% to 0.007% or is about 0.005%, and the second dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8% or is from 0.2% to 0.75% or is about 0.5%.

6. The method of claim 1, wherein the first dilution is a dilution of the test sample of from 0.001% to 0.009% (or 0.001%, 0.002%, 0.003%, 0.004%, 0.005%, 0.006%, 0.007%, 0.008% or 0.009%) or is from 0.002% to 0.008%, or is from 0.003% to 0.007% or is about 0.005%; and the second dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%.

7. The method of claim 1, wherein the first dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%; and
the second dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%.

8. The method of claim 1, wherein the first dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%; and
the second dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008%, or is from 0.003% to 0.007%, or is about 0.005%.

9. The method of claim 1, wherein the first dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%, and the second dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%.

10. The method of claim 1, wherein the first dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%, and the second dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008%, or is from 0.003% to 0.007%, or is about 0.005%.

11. The method of claim 1, wherein the detecting for the presence or the determining of the level of the dissociated first and second capture reagents is performed by PCR, mass spectrometry, nucleic acid sequencing, next-generation sequencing (NGS) or hybridization.

12. The method of claim 1, wherein the first aptamer and/or the second aptamer, independently, comprises at least one 5-position modified pyrimidine.

13. The method of claim 12, wherein the at least one 5-positon modified pyrimidine comprises a linker at the 5-position of the pyrimidine and a moiety attached to the linker.

14. The method of claim 13, wherein the linker is selected from amide linker, a carbonyl linker, a propynyl linker, an alkyne linker, an ester linker, a urea linker, a carbamate linker, a guanidine linker, an amidine linker, a sulfoxide linker, and a sulfone linker.

15. The method of claim 13, wherein the moiety is a hydrophobic moiety.

16. The method of claim 15, wherein the moiety is selected from the moieties of Groups I, II, III, IV, V, VII, VIII, IX, XI, XII, XIII, XV and XVI of FIG. 1.

17. The method of claim 15, wherein the moiety is selected from a naphthyl moiety, a benzyl moiety, a fluorobenzyl moiety, a tyrosyl moiety, an indole moiety a morpholino moiety, an isobutyl moiety, a 3,4-methylenedioxy benzyl moiety, a benzothiophenyl moiety, and a benzofuranyl moiety.

18. The method of claim 12, wherein the pyrimidine of the 5-position modified pyrimidine is a uridine, cytidine or thymidine.

19. (canceled)

20. The method of claim 1, wherein the third dilution sample is incubated separately from the first and second dilution samples to allow aptamer affinity complex formation of the third aptamer with its target molecule.

21. The method of 20, further comprising transferring the third dilution sample to the second mixture to form a third mixture, wherein the third aptamer affinity complex of the third dilution is captured on a solid support in the third mixture.

22. The method of claim 21, further comprising detecting for the presence of or determining the level of the third aptamer of the third aptamer affinity complex, or the presence or amount of the third aptamer affinity complex.

23. The method of claim 1, wherein the third dilution is a different dilution from the first dilution and the second dilution of the same test sample.

24. The method of claim 1, wherein the third dilution is a dilution of the test sample selected from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), from 15% to 30%, from 15% to 25%, about 20%; from 0.01% to 1% (or 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%), from 0.1% to 0.8%, from 0.2% to 0.75%, about 0.5%; and
from 0.001% to 0.009% (or 0.001%, 0.002%, 0.003%, 0.004%, 0.005%, 0.006%, 0.007%, 0.008% or 0.009%), or from 0.002% to 0.008%, from 0.003% to 0.007%, about 0.005%.

25. The method of claim 1, wherein the third aptamer comprises at least one 5-position modified pyrimidine.

26. The method of claim 25, wherein the at least one 5-positon modified pyrimidine comprises a linker at the 5-position of the pyrimidine and a moiety attached to the linker.

27. The method of claim 26, wherein the linker is selected from amide linker, a carbonyl linker, a propynyl linker, an alkyne linker, an ester linker, a urea linker, a carbamate linker, a guanidine linker, an amidine linker, a sulfoxide linker, and a sulfone linker.

28. The method of claim 26, wherein the moiety is a hydrophobic moiety.

29. The method of claim 28, wherein the moiety is selected from the moieties of Groups I, II, III, IV, V, VII, VIII, IX, XI, XII, XIII, XV and XVI of FIG. 1.

30. The method of claim 28, wherein the moiety is selected from a naphthyl moiety, a benzyl moiety, a fluorobenzyl moiety, a tyrosyl moiety, an indole moiety a morpholino moiety, an isobutyl moiety, a 3,4-methylenedioxy benzyl moiety, a benzothiophenyl moiety, and a benzofuranyl moiety.

31. The method of claim 25, wherein the pyrimidine of the 5-position modified pyrimidine is a uridine, cytidine or thymidine.

32.-75. (canceled)
